(19) United States
(12) Patent Application Publication
(10) Pub. No.: US 2026/0062376 A1
(43) Pub. Date: Mar. 5, 2026

(54) SQUARAMIDE DERIVATIVES AS CB1 ALLOSTERIC MODULATORS

(71) Applicant: RESEARCH TRIANGLE INSTITUTE, Research Triangle Park, NC (US)

(72) Inventors: Yanan ZHANG, Cary, NC (US); Thuy NGUYEN, Morrisville, NC (US)

(21) Appl. No.: 18/870,115

(22) PCT Filed: Jun. 2, 2023
(86) PCT No.: PCT/US2023/024264

§ 371 Date: Nov. 27, 2024

Related U.S. Application Data
(60) Provisional application No. 63/348,554, filed on Jun. 3, 2022.

(51) Int. Cl. C07C 225/22; A61K 31/137; A61K 31/381; A61K 31/397; A61K 31/402; A61K 31/426; A61K 31/4402; A61K 31/4409; A61K 31/4453; A61K 31/495; A61P 25/30; C07D 213/50; C07D 277/28; C07D 295/135; C07D 333/22

(52) U.S. Cl. CPC C07C 225/22; A61K 31/137; A61K 31/381; A61K 31/397; A61K 31/402; A61K 31/426; A61K 31/4402; A61K 31/4409; A61K 31/4453; A61K 31/495; A61P 25/30; C07D 213/50; C07D 277/28; C07D 295/135; C07D 333/22; C07C 2601/04

(57) ABSTRACT

Squaramide-based cannabinoid 1 receptor (CB1R) allosteric modulators are described. Exemplary analogs may provide improved potencies and pharmacokinetic properties. Methods of using the analogs to treat diseases mediated by CB1R, such as substance abuse and obesity, are described.

### GOVERNMENT INTEREST

This invention was made with government support from under Grant No. 5R01DA040693 awarded by the National Institutes of Health. The government has certain rights in the invention.

### FIELD OF THE INVENTION

The presently disclosed subject matter relates to squaramide-based cannabinoid 1 receptor (CB1R) allosteric modulator compounds, and to pharmaceutical compositions and uses thereof. Uses of the compounds include the modulation of CB1R activity and the treatment of diseases and conditions mediated by CB1R, such as obesity, substance abuse, alcohol addition, alcoholism, anxiety, depression, metabolic syndrome, stroke, hypotension, impaired fertility, cancer, inflammation, Parkinson's disease, paralytic ileus, and osteoporosis.

### BACKGROUND

According to the 2017 National Survey on Drug Use and Health, 18.7 million adults in the United States were affected with a substance abuse disorder. There are currently no FDA-approved medications for the treatment of craving to stimulants (e.g., cocaine, methamphetamine) and cannabis (marijuana). There are available medications for relapse prevention of other addictive substances (e.g., opioids, tobacco, and alcohol). However, while these medications may be effective for the treatment of withdrawal symptoms, the long-term abstinence rate is still low. For example, even with several medications for smoking cessation, the one-year abstinence rate is only about 20%, compared to about 10% for placebo. Accordingly, there is an unmet demand for medications that alleviate substance craving on a long-term basis.

The cannabinoid 1 and cannabinoid 2 receptors (CB1R and CB2R, respectively) belong to the Class A Rhodopsin-like superfamily of G protein-coupled receptors (GPCRs). CB1R is one of the most abundantly expressed receptors in the brain. See Matsuda et al., Nature 1990, 346, 561-564. CB1R plays a role in many physiological processes, such as pain, learning and memory, appetite and feeing behaviors, anxiety and depression. See Porter et al., Pharmacol. Ther. 2001, 90, 45-60; Harkany et al., Trends Pharmacol. Sci. 2007, 28, 83-92; and Kreitzer and Regehr, Curr. Opin. Neurobiol. 2002, 12, 324-330. As (−)-trans-Δ⁹-tetrahydrocannabinol (THC), the major phytocannabinoid found in marijuana, has been known for centuries to induce appetite and weight gains, as well as addiction, CB1R has been investigated to develop therapeutic interventions for obesity, metabolic disorders and substance abuse. See Van Gaal et al., Lancet 2005, 365, 1389-1397; Pi-Sunver et al., JAMA 2006, 295, 761-775; Scheen et al., Lancet 2006, 368, 1660-1672; Rosenstock et al., Diabetes Care 2008, 31, 2169-2176; Despres et al., Arterioscler. Thromb. Vasc. Biol. 2009, 29, 416-423; Steinberg and Foulds, Vasc. Health Risk Manag. 2007, 3, 307-311; and Huestis et al., Psychopharmacology (Berl) 2007, 194, 505-515. Other potential uses of CB1R antagonists/inverse agonists include the treatment of cancer, impaired fertility in women, stroke, hypotension, and intestinal hypomotility in paralytic ileus. See Pertwee and Thomas, “Therapeutic Applications for Agents that Act at CB1 and CB2 Receptors,” in The Cannabinoid Receptors, Reggio, Ed., Humana Press: 2009, pp. 361-392; and Youssif et al., European Journal of Medicinal Chemistry 2019, 177, 1-11. Unfortunately, rimonabant (also known as SR141716A), the first CB1R inverse agonist/antagonist that received FDA approval for the treatment of obesity in 2006, was subsequently withdrawn due to adverse effects, including suicidal ideation.

Accordingly, there is an ongoing need for additional compounds that may modulate CB1R activity to treat substance addiction, and other conditions that may be modulated via CB1R. For example, there is an ongoing need for additional CB1R modulator compounds that have reduced side effects, improved pharmacokinetic properties (e.g., metabolic stability), and improved potencies.

### SUMMARY

One embodiment of the present disclosure includes a compound of formula (I)

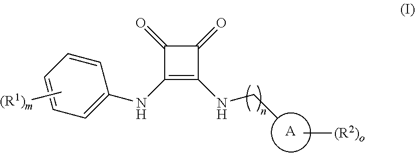

wherein
each R¹ is independently C₁-C₆ alkyl, C₂-C₆ alkenyl, C₂-C₆ alkynyl, halogen, C₁-C₆ haloalkyl, NO₂, CN, O—(C₁-C₆ alkyl), or N(R³)₂;
each R² is independently C₁-C₆ alkyl, C₂-C₆ alkenyl, C₂-C₆ alkynyl, halogen, C₁-C₆ haloalkyl, CN, NO₂, OH, O—(C₁-C₆ alkyl), O-(5- to 13-membered cycloalkyl), N(R³)₂, C(O)OH, C(O)C₁-C₆ alkyl, (C₁-C₈-alkyl)ₓ-(5- to 13-membered aryl)-(R⁴)ₚ, (C₁-C₈-alkyl)ₓ-(5- to 13-membered heterocyclyl containing one, two, or three heteroatoms selected from N, O, or S)—(R⁴)ₚ, (C₁-C₈-alkyl)ₓ-(5- to 13-membered heteroaryl containing one, two, or three heteroatoms selected from N, O, or S)—(R⁴)ₚ;
each R³ is independently H or C₁₋₆ alkyl; or two R³ groups and the nitrogen atom to which they are attached form a 5-7 atom heterocyclic ring which may contain one or two more additional heteroatoms selected from N, O, and S;
each R⁴ is independently H, halo, C₁₋₆ alkyl or N(R⁵)₂ wherein each R⁵ is independently H or C₁₋₆ alkyl or two R⁵ groups and the nitrogen atom to which they are attached form a 5-7 atom heterocyclic ring which may contain one or two more additional heteroatoms selected from N, O, and S;
each x independently is 0 or 1;
n is 0, 1, 2, 3, or 4;
m is 0, 1, 2, or 3; and
o is 0, 1, 2, or 3; and
p is 0, 1, 2, or 3,
or a pharmaceutically acceptable salt or solvate thereof.

In one aspect, Ring A is C₆ aryl, namely phenyl.

In one aspect, each R¹ is independently halo, C₁-C₄ alkyl, C₁-C₄ haloalkyl, CN, O—(C₁-C₆ alkyl), or N(R³)₂; each R² is independently halo, C₁-C₄ alkyl, C₁-C₄ haloalkyl, CN, NO₂, O—(C₁-C₆ alkyl), O-(5- to 13-membered cycloalkyl), N(R³)₂, C₁-C₈-alkyl)ₓ-(5- to 13-membered aryl), or (C₁-C₈-alkyl)ₓ-(5- to 13-membered heteroaryl), wherein each of aryl and heteroaryl is optionally substituted with one or more R⁵ groups; and wherein the heteroaryl contains one, two, or three heteroatoms selected from N, O, and S; each R³ is independently H or C₁₋₆ alkyl; or two R³ groups and the nitrogen atom to which they are attached form a 5-7 atom heterocyclic ring which may contain one or two more additional heteroatoms selected from N. O, and S; each R⁴ is independently H, halo, C₁₋₆ alkyl or N(R⁵)₂ wherein each R⁵ is independently H or C₁₋₆ alkyl or two R⁵ groups and the nitrogen atom to which they are attached form a 5-7 atom heterocyclic ring which may contain one or two more additional heteroatoms selected from N, O, and S; n is 0, 1, 2, 3, or 4; m is 1, 2, or 3; and o is 0, 1, 2, or 3, or a pharmaceutically acceptable salt or solvate thereof.

One embodiment of the present disclosure includes a compound of formula (II):

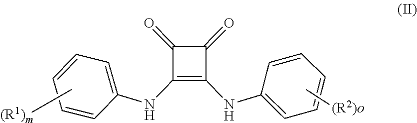

wherein
each R¹ is independently C₁-C₆ alkyl, C₂-C₆ alkenyl, C₂-C₆ alkynyl, halogen, C₁-C₆ haloalkyl, NO₂, CN, O—(C₁-C₆ alkyl), or N(R³)₂;
each R² is independently C₁-C₆ alkyl, C₂-C₆ alkenyl, C₂-C₆ alkynyl, halogen, C₁-C₆ haloalkyl, CN, O—(C₁-C₆ alkyl), N(R³)₂, C₁-C₈-alkyl)ₓ-(5- to 13-membered aryl), or (C₁-C₈-alkyl)ₓ-(5- to 13-membered heteroaryl), wherein each of aryl and heteroaryl is optionally substituted with one or more R⁴ groups, and wherein the heteroaryl contains one, two, or three heteroatoms selected from N, O, and S;
each R³ is independently H or C₁₋₆ alkyl; or two R³ groups and the nitrogen atom to which they are attached form a 5-7 atom heterocyclic ring which may contain one or two more additional heteroatoms selected from N, O, and S;
each R⁴ is independently H, halo, C₁₋₆ alkyl or N(R⁵)₂ wherein each R⁵ is independently H or C₁₋₆ alkyl;
m is 0, 1, 2, or 3; and
o is 0, 1, 2, or 3;
or a pharmaceutically acceptable salt or solvate thereof.

In one embodiment, when R² is other than pyridyl, two R⁵ groups and the nitrogen atom to which they are attached may form a 5-7 membered hetero ring, which may contain one or two more additional heteroatoms selected from N, O, and S, and which may contain one or more degrees of unsaturation.

In certain embodiments, Ring A is pyridinyl, thiophenyl, piperidinyl, or piperazinyl.

In one aspect, R¹ is halogen or CN. In one aspect, R¹ is halogen. In one aspect, R¹ is Cl.

In one aspect, n is 1 or 2. In one aspect, n is 2

In one aspect, o is 0.

In one aspect, o is at least 1 and each R² is selected from the group consisting of C₁₋₆alkyl, halogen, O(C₁₋₆ alkyl), C₁₋₆ haloalkyl, N(C₁₋₆ alkyl)₂, C(O)C₁₋₆ alkyl, OH, unsubstituted phenyl, phenyl substituted with halogen, unsubstituted pyridine, pyridine substituted with pyrroline, unsubstituted thiazole, unsubstituted thiophene, unsubstituted phenyl, and phenyl substituted with halogen.

In one aspect, o is 1 or 2, and each R² is selected from the group consisting of C₁₋₆alkyl, halogen, O(C₁₋₆ alkyl), N(C₁₋₆ alkyl)₂, C(O)C₁₋₆ alkyl, unsubstituted phenyl, and phenyl substituted with halogen.

In one aspect, R³ is CH₃.

In one aspect, R⁴ is H, halo or N(R⁵)₂, where the two R⁵ groups and the nitrogen to which they are attached form a pyrrolidine.

In one aspect, x is 0.

One embodiment of the present disclosure includes a compound selected from:
3-(Benzylamino)-4-[(4-chlorophenyl)amino]cyclobut-3-ene-1,2-dione (9);
3-[(4-Chlorophenyl)amino]-4-[(2-phenylethyl)amino]cyclobut-3-ene-1,2-dione (10);
3-[(4-Chlorophenyl)amino]-4-[(3-phenylpropyl)amino]cyclobut-3-ene-1,2-dione (11);
3-[(4-Chlorophenyl)amino]-4-{[2-(4-chlorophenyl)ethyl]amino}cyclobut-3-ene-1,2-dione (12);
3-[(4-Chlorophenyl)amino]-4-{[2-(3-methylphenyl)ethyl]amino}cyclobut-3-ene-1,2-dione (13);
3-[(4-Chlorophenyl)amino]-4-{[2-(4-methylphenyl)ethyl]amino}cyclobut-3-ene-1,2-dione (14);
3-[(4-Chlorophenyl)amino]-4-{[2-(2-methoxyphenyl)ethyl]amino}cyclobut-3-ene-1,2-dione (15);
3-[(4-Chlorophenyl)amino]-4-{[2-(3-methoxyphenyl)ethyl]amino}cyclobut-3-ene-1,2-dione (16);
3-[(4-Chlorophenyl)amino]-4-{[2-(4-methoxyphenyl)ethyl]amino}cyclobut-3-ene-1,2-dione (17);
3-[(4-Chlorophenyl)amino]-4-({2-[2-(trifluoromethyl)phenyl]ethyl}amino)cyclobut-3-ene-1,2-dione (18);
3-[(4-Chlorophenyl)amino]-4-({2-[3-(trifluoromethyl)phenyl]ethyl}amino)cyclobut-3-ene-1,2-dione (19);
3-[(4-Chlorophenyl)amino]-4-({2-[4-(trifluoromethyl)phenyl]ethyl}amino)cyclobut-3-ene-1,2-dione (20);
3-[(4-Chlorophenyl)amino]-4-{[2-(4-fluorophenyl)ethyl]amino}cyclobut-3-ene-1,2-dione (21);
3-[(4-Chlorophenyl)amino]-4-{[2-(2-chlorophenyl)ethyl]amino}cyclobut-3-ene-1,2-dione (22);
3-[(4-Chlorophenyl)amino]-4-{[2-(3-chlorophenyl)ethyl]amino}cyclobut-3-ene-1,2-dione (6);
3-[(4-Chlorophenyl)amino]-4-{[2-(2-fluorophenyl)ethyl]amino}cyclobut-3-ene-1,2-dione (23);
3-[(4-Chlorophenyl)amino]-4-{[2-(3-fluorophenyl)ethyl]amino}cyclobut-3-ene-1,2-dione (24);
3-[(4-Chlorophenyl)amino]-4-({2-[3-(dimethylamino)phenyl]ethyl}amino)cyclobut-3-ene-1,2-dione (25);
3-[(4-Chlorophenyl)amino]-4-({2-[4-(dimethylamino)phenyl]ethyl}amino)cyclobut-3-ene-1,2-dione (26);
4-[(2-{[2-(3-Chlorophenyl)ethyl]amino}-3,4-dioxocyclobut-1-en-1-yl)amino]benzonitrile (27);
3-(Biphenyl-3-ylamino)-4-[(4-chlorophenyl)amino]cyclobut-3-ene-1,2-dione (28);
3-[(4-Chlorophenyl)amino]-4-{[3-(thiophen-3-yl)phenyl]amino}cyclobut-3-ene-1,2-dione (31);
3-[(4-Chlorophenyl)amino]-4-[(4′-fluorobiphenyl-3-yl)amino]cyclobut-3-ene-1,2-dione (32);
3-(Biphenyl-4-ylamino)-4-[(4-chlorophenyl)amino]cyclobut-3-ene-1,2-dione (33);
3-[(4-Chlorophenyl)amino]-4-{[2-(2-methylphenyl)ethyl]amino}cyclobut-3-ene-1,2-dione;
3-[(4-Chlorophenyl)amino]-4-{[2-(pyridin-2-yl)ethyl]amino}cyclobut-3-ene-1,2-dione;
3-[(4-Chlorophenyl)amino]-4-{[2-(pyridin-4-yl)ethyl]amino}cyclobut-3-ene-1,2-dione;
3-[(4-Chlorophenyl)amino]-4-{[2-(4-bromophenyl)ethyl]amino}cyclobut-3-ene-1,2-dione;
3-[(4-Chlorophenyl)amino]-4-{[2-(thiophen-2-yl)ethyl]amino}cyclobut-3-ene-1,2-dione;
3-{[2-(A-acetylphenyl)ethyl]amino}-4-[(4-chlorophenyl)amino]cyclobut-3-ene-1,2-dione;
3-[(4-Chlorophenyl)amino]-4-{[2-(3,4-dichlorophenyl)ethyl]amino}cyclobut-3-ene-1,2-dione;
3-[(4-Chlorophenyl)amino]-4-{[2-(3,4-dimethylphenyl)ethyl]amino}cyclobut-3-ene-1,2-dione;
3-[(4-Chlorophenyl)amino]-4-{[2-(3-hydroxyphenyl)ethyl]amino}cyclobut-3-ene-1,2-dione;
3-[(4-Chlorophenyl)amino]-4-{[2-(2,4-dichlorophenyl)ethyl]amino}cyclobut-3-ene-1,2-dione; 3-[(4-Chlorophenyl)amino]-4-{[2-(piperidin-1-yl)ethyl]amino}cyclobut-3-ene-1,2-dione;
3-[(4-Chlorophenyl)amino]-4-({2-[4-(4-chlorophenyl)piperazin-1-yl]ethyl}amino)cyclobut-3-ene-1,2-dione;
3-[(4-Chlorophenyl)amino]-4-{[2-(2,4-difluorophenyl)ethyl]amino}cyclobut-3-ene-1,2-dione; and
3-[(4-Chlorophenyl)amino]-4-({2-[4-(diethylamino)phenyl]ethyl}amino)cyclobut-3-ene-1,2-dione,
or a pharmaceutically acceptable salt thereof.

One embodiment of the present disclosure includes a compound selected from: Compound 6, 12, 13, 14, or a pharmaceutically acceptable salt thereof.

One embodiment of the present disclosure includes a pharmaceutical composition comprising a compound of the present disclosure and a pharmaceutically acceptable carrier.

A method of treating a cannabinoid 1 receptor (CB1R)-mediated disease or condition in a subject in need of treatment thereof, the method comprising administering to said subject a therapeutically effective amount of a compound of the present disclosure.

In one aspect, the subject is a mammal, optionally a human.

In one aspect, the disease or condition is selected from the group consisting of addiction, obesity, cancer, pain, female infertility, memory loss, cognitive dysfunction, Parkinson's disease, dyskinesia, tardive dyskinesia, Alzheimer's disease, amyotrophic lateral sclerosis (ALS), Tourette's Syndrome, stroke, atherosclerosis, hypotension, intestinal hypoactivity in paralytic ileus, inflammation, osteoporosis, hypercholesterolemia, dyslipidemia, diabetes, retinopathy, glaucoma, anxiety, depression and other mood disorders, gastrointestinal disorders, and metabolic disorders.

In one aspect, the disease is obesity or addiction, optionally wherein the addiction is selected from cocaine addiction, opioid addiction, amphetamine addiction, cannabinoid addition, tobacco addiction, and alcohol addiction.

One embodiment of the present disclosure includes a method for inhibiting substance abuse, addiction, addictive behavior, or a symptom, behavior, or condition associated with addiction, the method comprising administering to a subject in need thereof a therapeutically effective amount of a compound of the present disclosure.

In one aspect, the addiction is selected from cocaine addiction, opioid addiction, amphetamine addiction, cannabinoid addition, tobacco addiction, and alcohol addiction.

In one aspect, the administration of a compound of the present disclosure prevents drug taking.

In one aspect, the administration of a compound of the present disclosure prevents or inhibits relapse.

One embodiment of the present disclosure includes a method of modulating the activity of cannabinoid 1 receptor (CB1R), wherein the method comprises contacting a sample comprising CB1R with a compound of the present disclosure.

One embodiment of the present disclosure includes a compound of the present disclosure for use in medicine.

One embodiment of the present disclosure includes a compound of the present disclosure for the manufacture of a medicament for the treatment of one or more diseases or disorder of addiction, obesity, cancer, pain, female infertility, memory loss, cognitive dysfunction, Parkinson's disease, dyskinesia, tardive dyskinesia, Alzheimer's disease, amyotrophic lateral sclerosis (ALS), Tourette's Syndrome, stroke, atherosclerosis, hypotension, intestinal hypoactivity in paralytic ileus, inflammation, osteoporosis, hypercholesterolemia, dyslipidemia, diabetes, retinopathy, glaucoma, anxiety, depression and other mood disorders, gastrointestinal disorders, and metabolic disorders.

In one aspect, the disease or disorder is obesity or addiction, optionally wherein the addiction is selected from cocaine addiction, opioid addiction, amphetamine addiction, cannabinoid addition, tobacco addiction, and alcohol addiction.

One embodiment of the present disclosure includes use of a compound of the present disclosure for the treatment of one or more diseases or disorders of addiction, obesity, cancer, pain, female infertility, memory loss, cognitive dysfunction, Parkinson's disease, dyskinesia, tardive dyskinesia, Alzheimer's disease, amyotrophic lateral sclerosis (ALS), Tourette's Syndrome, stroke, atherosclerosis, hypotension, intestinal hypoactivity in paralytic ileus, inflammation, osteoporosis, hypercholesterolemia, dyslipidemia, diabetes, retinopathy, glaucoma, anxiety, depression and other mood disorders, gastrointestinal disorders, and metabolic disorders.

In one aspect, the disease or disorder is obesity or addiction, optionally wherein the addiction is selected from cocaine addiction, opioid addiction, amphetamine addiction, cannabinoid addition, tobacco addiction, and alcohol addiction.

One or more aspects and embodiments may be incorporated in a different embodiment although not specifically described. That is, all aspects and embodiments may be combined in any way or combination.

### BRIEF DESCRIPTION OF THE DRAWINGS

The presently disclosed subject matter may be better understood by referring to the following FIGURES. The components in the figures are not necessarily to scale, emphasis instead being placed upon illustrating the principles of the presently disclosed subject matter. The drawings are not intended to limit the scope of this presently disclosed subject matter, which is set forth with particularity in the claims as appended or as subsequently amended, but merely to clarify and exemplify the presently disclosed subject matter. For a more complete understanding of the presently disclosed subject matter, reference is now made to the below drawings.

FIG. 1 is a graphical illustration demonstrating that (A) Compound 6 at 5.6 mg/kg (i.p.) was effective to attenuate cocaine-reseeking behavior in rats; and (B) Compound 6 did not affect locomotion at 5.6 mg/kg (i.p.).

### DETAILED DESCRIPTION

The presently disclosed subject matter will now be described more fully hereinafter with reference to the accompanying Examples, in which representative embodiments are shown. The presently disclosed subject matter may, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist, unless as otherwise specifically indicated.

### Definitions

Unless otherwise indicated, all numbers expressing quantities of ingredients, reaction conditions, and so forth used in the specification and claims are to be understood as being modified in all instances by the term “about,” unless stated otherwise. Accordingly, unless indicated to the contrary, the numerical parameters set forth in the present specification and attached claims are approximations that may vary depending upon the desired properties sought to be obtained by the presently disclosed subject matter. The term “about”, as used herein when referring to a measurable value such as an amount of weight, molar equivalents, time, temperature, etc. is meant to encompass in one example variations of ±20% or ±10%, in another example ±5%, in another example ±1%, and in yet another example ±0.1% from the specified amount, as such variations are appropriate to perform the disclosed methods.

As used herein the term “alkyl” refers to C₁-C₂₀ inclusive, linear (i.e., “straight-chain”), branched, saturated, partially saturated, and fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁-C₈ alkyl), e.g., 1, 2, 3, 4, 5, 6, 7, or 8 carbon atoms. In some embodiments, “lower alkyl” may refer to C₁₋₆ or C₁₋₅ alkyl groups.

Alkyl groups may optionally be substituted (a “substituted alkyl”) with one or more substituents, which may be the same or different. The term “substituent” includes but is not limited to alkyl, halo, haloalkyl, nitro, cyano, amino, arylamino, acyl, hydroxyl, aryloxyl, alkoxyl, alkylthio, arylthio, aralkyloxyl, aralkylthio, carboxyl, alkoxycarbonyl, oxo, and cycloalkyl. There may be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, halogen, haloalkyl, aryl, substituted aryl, alkoxyl, hydroxyl, nitro, cyano, amino, alkylamino, dialkylamino, ester, acyl, amide, sulfonyl, sulfate, and mercapto.

The term “alkenyl” refers to an alkyl group as defined above including at least one carbon-carbon double bond. Exemplary alkenyl groups include, but are not limited to, ethenyl, propenyl, butenyl, pentenyl, hexenyl, octenyl, butadienyl, and allenyl groups. Alkenyl groups may optionally be substituted with one or more alkyl group substitutents, which may be the same or different, including, but not limited to alkyl (saturated or unsaturated), substituted alkyl (e.g., halo-substituted and perhalo-substituted alkyl, such as but not limited to, —CF₃), cycloalkyl, halo, nitro, hydroxyl, carbonyl, carboxyl, acyl, alkoxyl, aryloxyl, aralkoxyl, thioalkyl, thioaryl, thioaralkyl, amino (e.g., aminoalkyl, aminodialkyl, aminoaryl, etc.), sulfonyl, and sulfinyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. In some embodiments, the cycloalkyl ring system comprises between 3 and 6 carbon atoms. The cycloalkyl group may be optionally partially unsaturated. The cycloalkyl group also may be optionally substituted with a substituent as defined herein. Representative monocyclic cycloalkyl rings include, but are not limited to, cyclopropyl, cyclobutyl, cyclopentyl, cyclohexyl, cycloheptyl, and the like. Further, the cycloalkyl group may be optionally attached though a linking group, such as an alkylene group as defined hereinbelow, for example, methylene, ethylene, propylene, and the like. In such cases, the cycloalkyl group may be referred to as, for example, cyclopropylmethyl, cyclobutylmethyl, and the like. Additionally, multicyclic cycloalkyl rings include adamantyl, octahydronaphthyl, decalin, camphor, camphane, and noradamantyl.

Thus, as used herein, the term “substituted cycloalkyl” includes cycloalkyl groups, as defined herein, in which one or more atoms or functional groups of the cycloalkyl group are replaced with another atom or functional group, including for example, alkyl, halogen, haloalkyl, aryl, substituted aryl, alkoxyl, hydroxyl, nitro, cyano, amino, alkylamino, dialkylamino, ester, acyl, amide, sulfonyl, sulfate, and mercapto.

The term “aryl” is used herein to refer to an aromatic substituent that may be a single aromatic ring, or multiple aromatic rings that are fused together, linked covalently, or linked to a common group, such as, but not limited to, a methylene or ethylene moiety. The common linking group also may be a carbonyl, as in benzophenone, or oxygen, as in diphenylether, or nitrogen, as in diphenylamine. The term “aryl” specifically encompasses heterocyclic aromatic compounds (i.e., “heteroaryl”). The aromatic ring(s) may comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group may be optionally substituted (a “substituted aryl”) with one or more substituents, which may be the same or different, and includes alkyl, aryl, halogen, haloalkyl, aralkyl, hydroxyl, alkoxyl, aryloxyl, aralkyloxyl, carboxyl, acyl, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ may each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, halogen, haloalkyl, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyridine, imidazole, benzimidazole, isothiazole, isoxazole, pyrazole, pyrazine, triazine, thiazole, pyrimidine, quinoline, isoquinoline, indole, carbazole, napthyl, and the like.

“Heterocyclic”, “heterocycle”, or “heterocyclo” as used herein alone or as part of another group, refers to an aliphatic (e.g., fully or partially saturated heterocyclo) monocyclic- or a bicyclic-ring system comprising one or more heteroatoms (e.g., 1, 2, or 3 heteroatoms selected from oxygen, sulfur, and substituted or unsubstituted nitrogen) inserted along the cyclic alkyl or aryl carbon chain. Monocyclic ring systems are exemplified by any 5- or 6-membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, ethylene oxide, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydropyran, tetrahydrothiophene (also known as thiolane), tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, carbazole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings include quaternized derivatives thereof and may be optionally substituted with one or more alkyl and/or aryl group substituents.

“Substituted heterocyclic” as used herein refers to a heterocyclic group wherein one or more hydrogen atom is replaced by a substitutent.

The term “N-heterocycle” refers to a heterocycle wherein at least one of the heteroatoms is a nitrogen atom. Examples of N-heterocycles include, but are not limited to, azetidine, pyrrolidine, pyrrole, pyrroline, pyrazole, pyrazoline, pyrazolidine, piperidine, pyridine, piperazine, pyrazine, pyrimidine, pyridazine, morpholine, imidazole, benzimidazole, imidazoline, imidazolidine, indole, carbazole, quinoline, isoquinoline, oxazole, thiazole, isothiazole, and thiazine.

“Substituted N-heterocycle” refers to an N-heterocycle wherein one or more hydrogen is replaced by a substituent.

The term “heteroaryl” refers to an aromatic monocyclic- or a bicyclic-ring system (a fused, bridged or spirocyclic ring system) comprising one or more heteroatoms (e.g., 1, 2, or 3 heteroatoms selected from oxygen, sulfur, and substituted or unsubstituted nitrogen, wherein N-oxides, sulfur oxides and dioxides are permissible heteroatom substitutions) inserted along the cyclic aryl carbon chain. In some embodiments, the monocyclic heteroaryl group is a five to seven membered aromatic ring. Representative heteroaryl groups include, but are not limited to, furan, thiophene, pyrrole, imidazole, pyrazole, triazole, tetrazole, oxazole, isoxazole, oxadiazole, thiaciazole, isothiazole, pyridine, pyridazine, pyrazine, pyrimidine, quinoline, isoquinoline, benzofuran, benzoxazole, benzothiophene, indole, indazole, benzimidazole, imidazopyridine, pyrazolopyrindine, and pyrazolopyrimidine.

The term “substituted heteroaryl” refers to a heteroaryl group as defined herein wherein one or more hydrogen atoms is replaced by a substituent.

“Aralkyl” refers to an aryl-alkyl- or an -alkyl-aryl group wherein aryl and alkyl are as previously described and may include substituted aryl and substituted alkyl. Thus, “substituted aralkyl” may refer to an aralkyl group comprising one or more substituents. Exemplary aralkyl groups include benzyl, phenylethyl, and naphthylmethyl.

“Alkylene” may refer to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group may be straight or branched. The alkylene group also may be optionally unsaturated (i.e., include alkene or alkyne groups) and/or substituted with one or more “alkyl group substituents.” There may be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group may have about 2 to about 3 carbon atoms and may further have 6-20 carbons.

“Arylene” refers to a bivalent aryl group, which may be substituted or unsubstituted.

The term “aralkylene” refers to a bivalent group that comprises a combination of alkylene and arylene groups (e.g., -arylene-alkylene-, alkylene-arylene-alkylene-, arylene-alkylene-arylene-, etc.).

Similarly, the terms “cycloalkylene”, “heterocycloalkylene,” and “heteroarylene” refer to bivalent cycloalkyl, heterocyclic, and heteroaryl groups, which may optionally be substituted with one or more substitutents.

As used herein, the term “acyl” refers to an organic carboxylic acid group wherein the —OH of the carboxylic acid group has been replaced with another substituent. Thus, an acyl group may be represented by RC(═O)—, wherein R is an alkyl, substituted alkyl, aralkyl, substituted aralkyl, aryl or substituted aryl group as defined herein. As such, the term “acyl” specifically includes arylacyl groups, such as a phenacyl group. Specific examples of acyl groups include acetyl (i.e., —C(═O)CH₃) and benzoyl.

“Alkoxyl” refers to an alkyl-O— group wherein alkyl is as previously described, including substituted alkyl. The term “alkoxyl” as used herein may refer to, for example, methoxyl, ethoxyl, propoxyl, isopropoxyl, butoxyl, t-butoxyl, and pentoxyl. The terms “oxyalkyl” and “alkoxy” may be used interchangably with “alkoxyl”.

“Aryloxyl” and “aryloxy” refer to an aryl-O— group wherein the aryl group is as previously described, including a substituted aryl. The term “aryloxyl” as used herein may refer to phenyloxyl or hexyloxyl, and to alkyl, substituted alkyl, or alkoxyl substituted phenyloxyl or hexyloxyl.

“Aralkyloxyl” or “aralkoxy” refer to an aralkyl-O— group wherein the aralkyl group is as previously described. An exemplary aralkyloxyl group is benzyloxyl.

The term “carbonyl” refers to the group —C(═O)—. The term “carbonyl carbon” refers to a carbon atom of a carbonyl group. Other groups such as, but not limited to, acyl groups, anhydrides, aldehydes, esters, lactones, amides, ketones, carbonates, and carboxylic acids, include a carbonyl group.

The terms “carboxyl” and “carboxylic acid” refer to the —C(═O)OH or —C(═O)O⁻ group.

The term “acid chloride” may refer to the —C(═O)Cl group.

The terms “halo” or “halogen” as used herein refer to fluoro, chloro, bromo, and iodo groups.

The term “haloalkyl” refers to an alkyl group as defined herein substituted by one or more halo groups. The term “perhaloalkyl” refers to an alkyl group as defined herein wherein all C—H bonds are replaced by carbon-halogen bonds. The term “perfluoroalkyl” refers to an alkyl group wherein all C—H bonds are replaced by C—F bonds. An exemplary perfluoroalkyl group is trifluoromethyl (—CF₃).

The term “sulfonyl” refers to the —S(═O)₂R group, wherein R is alkyl, substituted alkyl, aralkyl, substituted aralkyl, aryl, or substituted aryl. The term “alkylsulfonyl” refers to the —S(═O)₂R group, wherein R is alkyl or substituted alkyl. In some embodiments, the sulfonyl group is —S(═O)₂CH₃.

The term “ester” refers to the R′—O—C(═O)— group, wherein the carbonyl carbon is attached to another carbon atom and wherein R′ is alkyl, cycloalkyl, aralkyl, or aryl, wherein the alkyl, cycloalkyl, aralkyl, or aryl are optionally substituted. The term “esterifying” may refer to forming an ester by contacting a compound containing a carboxylic acid or derivative thereof (e.g., an acid chloride) and a compound containing a hydroxyl group (e.g., an alcohol or a phenol).

The term “amide” refers to a compound comprising the structure R′—NR″—C(═O)—R, wherein R is alkyl, substituted alkyl, aralkyl, substituted aralkyl, aryl or substituted aryl, and wherein R′ and R″ are independently hydrogen, alkyl, aralkyl, or aryl, wherein the alkyl, aralkyl, or aryl are optionally substituted. In some embodiments, R′ is alkyl, substituted alkyl, aralkyl, substituted aralkyl, aryl, or substituted aryl.

The term “urea” as used herein refers to a compound comprising the structure R—NR′—C(═O)—NR′—R, wherein each R is independently alkyl, substituted alkyl, aralkyl, substituted aralkyl, aryl, or substituted aryl, and wherein each R′ is independently H, alkyl, substituted alkyl, aralkyl, substituted aralkyl, aryl, or substituted aryl.

The term “amine” refers to a molecule having the formula N(R)₃, or a protonated form thereof, wherein each R is independently H, alkyl, substituted alkyl, aryl, substituted aryl, aralkyl, substituted aralkyl, or wherein two R groups together form an alkylene or arylene group. The term “primary amine” refers to an amine wherein at least two R groups are H. The term “secondary amine” refers to an amine wherein only one R group is H. The term “alkylamine” may refer to an amine wherein two R groups are H and the other R group is alkyl or substituted alkyl. “Dialkylamine” may refer to an amine where two R groups are alkyl. “Arylamine” may refer to an amine wherein one R group is aryl. Amines may also be protonated, i.e., have the formula [NH(R)₃]⁺.

The term “amino” refers to the group —N(R)₂ wherein each R is independently H, alkyl, substituted alkyl, aryl, substituted aryl, aralkyl, or substituted aralkyl. The terms “aminoalkyl” and “alkylamino” may refer to the group —N(R)₂ wherein each R is H, alkyl or substituted alkyl, and wherein at least one R is alkyl or substituted alkyl. The term “dialkylamino” refers to an aminoalkyl group where both R groups are alkyl or substituted alkyl, which may be the same or different.

The terms “acylamino” and “aminoacyl” refer to the —N(R)—C(═O)R′ group, wherein R is selected from H, alkyl, substituted alkyl, aralkyl, substituted aralkyl, aryl, and substituted aryl, and wherein R′ is selected from alkyl, substituted alkyl, aralkyl, substituted aralkyl, aryl, and substituted aryl.

The term “cyano” refers to the —C≡N group.

The terms “hydroxyl” and “hydroxy” refer to the —OH group.

The terms “mercapto” and “thiol” refer to the —SH group.

The term “oxo” refers to a compound described previously herein wherein a carbon atom is replaced by an oxygen atom.

The term “nitro” refers to the —NO₂ group.

The term “thioalkyl” may refer to the group —SR, wherein R is selected from H, alkyl, substituted alkyl, aralkyl, substituted aralkyl, aryl, and substituted aryl. Similarly, the terms “thioaralkyl” and “thioaryl” refer to —SR groups wherein R is aralkyl and aryl, respectively.

The terms “treatment” and “treating” and the like as used herein refers to any treatment of a disease and/or condition in an animal or mammal, particularly a human, and includes inhibiting the disease, disorder and/or condition, i.e., arresting its development; and relieving the disease, disorder and/or condition, i.e., causing regression of the disease, disorder and/or condition.

The phrase “protecting group” as used herein includes any suitable protecting group; “protected form” refers to a substituent in which an atom such as hydrogen has been removed and replaced with a corresponding protecting group. Protecting groups are known. See generally T. H. Greene and P. G. M. Wuts, Protective Groups in Organic Synthesis, 3rd edition, John Wiley & Sons, New York (1999). Examples include but are not limited to: hydroxy protecting groups (for producing the protected form of hydroxy); carboxy protecting groups (for producing the protected form of carboxylic acid); amino-protecting groups (for producing the protected form of amino); sulfhydryl protecting groups (for producing the protected form of sulfhydryl); etc. Particular examples include but are not limited to: benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl (trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, acetyl (Ac), benzoyl (Bn), and trimethylsilyl (TMS), and the like; formyl, acetyl, benzoyl, pivaloyl, t-butylacetyl, phenylsulfonyl, benzyl, t-butyloxycarbonyl (Boc), and benzyloxycarbonyl (Cbz) and the like; and hemithioacetals such as 1-ethoxyethyl and methoxymethyl, thioesters, or thiocarbonates and the like.

The term “allosteric modulator” as used herein refers to a compound (or “ligand”) that binds to a site on a macromolecule (e.g., a receptor) that is distinct from the orthosteric site (i.e., the primary binding site of the macromolecule). Allosteric modulators may indirectly influence the effects of an orthosteric or primary ligand that binds at the orthosteric site. For example, an allosteric modulator of the CB1 receptor may bind to the receptor at the site distinct to the orthosteric site(s) leading to a change in receptor conformation. As a result, interactive properties of the receptor with respect to orthosteric ligand(s) and cellular host environment may be modified in either a positive or negative direction, respectively referred to as positive allosteric modulators (“PAMs”) and negative allosteric modulators (“NAMs”). Allosteric modulators may exhibit the following pharmacological properties: (i) affinity modulation, where the resulting conformation may alter either association or dissociation rate of an orthosleric ligand; (ii) efficacy modulation, where the allosteric effect may modify intracellular response and lead to a change in the signaling capacity of the orthosteric ligand; and/or (iii) agonism/inverse agonism, where the allosteric modulator may perturb receptor signaling in either a positive or negative direction, irrespective of presence of orthosteric modulator.

Preclinical and clinical studies suggest that the blockade of CB1R is a promising strategy for the treatment of many common drugs of abuse, as well as a number of other conditions, including for example, but not limited to, obesity, anxiety, cancer, inflammation, Parkinson's disease, osteoporosis, female infertility, metabolic disorders, pain, stroke, hypotension, and intestinal hypoactivity. Unfortunately, to date, psychiatric side effects such as depression, anxiety, or even suicidal ideation, have restricted the use of CB1R antagonist/inverse agonists in the clinic.

Despite this setback, CB1R continues to be a target for drug development and various strategies have been explored to overcome the psychiatric adverse effects of CB1R signaling while preserving beneficial therapeutic effects. Like many GPCRs, CB1R displays a high level of constitutive activity in the absence of exogenous ligands in both neurons (see Pan et al., Mol. Pharmacol. 1998, 54, 1064-1072; and Hillard et al., FEBS Lett. 1999, 459, 277-281) and non-neuronal cells. See Bouaboula et al., J. Biol. Chem. 1997, 272, 22330-22339. As the constitutive activity is important to maintain cellular homeostasis, the adverse effects of the CB1R antagonist/inverse agonist rimonabant are thought to be derived from its CB1R inverse agonism that reduces CB1R basal tone. Therefore, it has been postulated that neutral antagonists which attenuate CB1R signaling in overactive conditions but leave the CB1R basal level unchanged could have fewer side effects. See Greasley and Clapham, Eur. J. Pharmacol. 2006, 553, 1-9. Peripherally restricted antagonists which do not cross the blood-brain barrier have also shown promising therapeutic efficacy in the treatment of obesity and diabetes without the liability of the central nervous system (CNS) side effects. See Chorvat, Bioorg. Med. Chem. Lett. 2013, 23, 4751-4760.

In addition, the discovery of CB1R allosteric binding sites has offered a promising alternative approach to modulate CB1R signaling for therapeutic benefits. Allosteric modulators target CB1R at the allosteric binding site, offering several advantages to orthosteric ligands, such as better receptor subtype selectivity, lower risk of overdosing due to the “ceiling” effect, and more transient pharmacological effects as a result of their dependence on the presence of endocannabinoids. See Nguyen et al., Med. Res. Rev. 2017, 37, 441-474.

The structures of previously studied CB1R negative allosteric modulators, Org27569 (Known Compound 1) and PSNCBAM-1 (Known Compound 2), are shown in below. See German et al., J. Med. Chem. 2014, 57, 7758-7769; and Nguyen et al., Bioorg. Med. Chem. 2015, 23, 2195-2203. Known Compound 2, for example, exhibits positive binding cooperativity with CP55,940, a cannabinoid receptor agonist that mimics the effects of THC; reduces the efficacy of agonists in several functional assays; and reduces food intake and body weight in rats. See Horswill et al., Br. J. Pharmacol. 2007, 152, 805-814.

Structure-activity relationship (SAR) efforts on PSNCBAM-1/Known Compound 2 have indicated that the pyrrolidinyl ring is not required for CB1R modulatory activity and that the pyridinyl ring may be replaced with substituted phenyl rings or five-membered heterocycles, such as in RTICBM-229 (Known Compound 5), also shown, which exhibits greater potency than Known Compound 2 in the [³⁵S]GTPγS binding assay and a higher maximum binding level in the [³H]CP55,940 binding assay. See German et al., J. Med. Chem. 2014, 57, 7758-7769; Nguyen et al., J. Med. Chem. 2017, 60, 7410-7424; and Nguyen et al., ACS Chem. Neurosci. 2019, 10, 518-527.

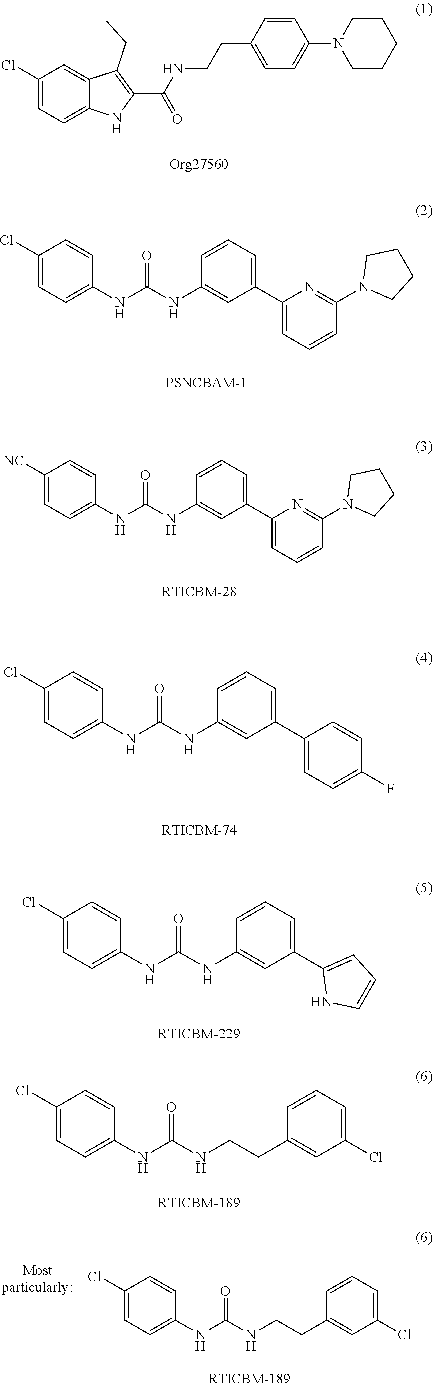

Structures of representative indole-based and diarylurea-based CB1 allosteric modulators.

Efforts to optimize diaryl urea-based Known Compound 2 also led to compound RT-CBM-74 (Known Compound 4). Known Compound 4 attenuates prime-induced restatement of cocaine seeking. RTICBM-28 (Known Compound 3), in which the chloro group in the outer phenyl ring of Known Compound 2 is replaced by cyano, reduces THC's potency in drug discrimination, demonstrating the therapeutic potential of these CB1R allosteric modulators for the treatment of the relapse of cocaine addiction (see Nguyen et al., J. Med. Chem. 2017, 60, 7410-7424) and THC dependence. See Gamage et al., Neuropharmacology 2017, 125, 365-375. Overall, SAR of the outer phenyl ring indicated that the 4-position favors electron withdrawing functionalities. See German et al., J. Med. Chem. 2014, 57, 7758-7769.

The subject matter of the present disclosure is based in part on further efforts to extend the understanding of the diaryl urea-based scaffold of Known Compound 2, by structural optimization at the middle phenyl ring depicted in the previously studied compounds, and the understanding of the aryl-alkyl urea-based scaffold of Known Compound 6 (RTICBM-189). RTICBM-189 (6), in which the phenyl group in the center if Known Compound 2 is replaced by an ethylene group, at 10 mg/kg attenuates drug-induced restatement of cocaine seeking in rats. See Nugyen et al., J. Med. Chem. 2022, 65, 257-270. SAR of the central phenyl ring indicated that an aromatic ring is not needed and an alkyl linker is tolerated.

In the present novel series of CB1 allosteric modulators, the urea functionality is replaced with a squaramide moiety, a group which has not been explored fully. This squaramide is either connected to an aliphatic (e.g ethylene) or an aromatic group (e.g. phenyl) on the depicted right hand side.

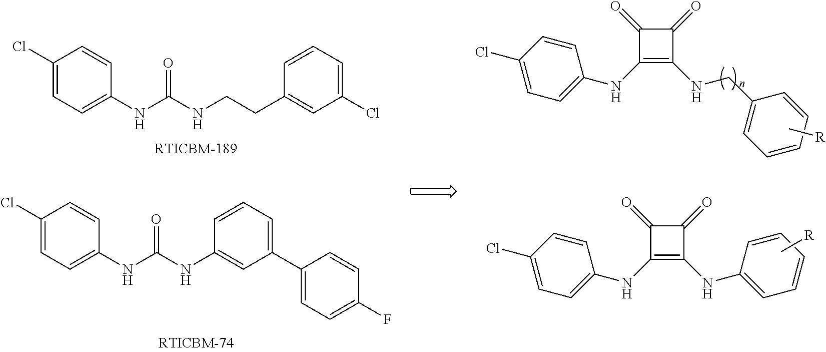

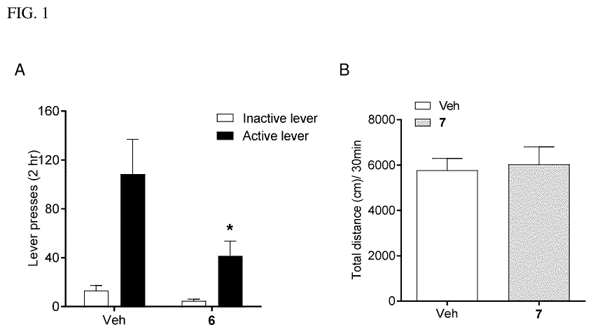

Replacement of the urea functionality with squaramide led to a series with distinct chemical structures. These novel compounds possess comparable in vitro activities in the calcium mobilization and [³⁵S]GTPγS binding assays (Tables 1 and 2). Compound 7 has better metabolic stability than PSNCBAM-1 (t_{1/2}: 40 min vs. 13 min in rat liver microsomes) and demonstrated better in vivo efficacy in attenuating cocaine reseeking behavior in rats at a low dose of 5.6 mg/kg i.p. (FIG. 1A) without affecting locomotion (FIG. 1B). In summary, this novel series of CB₁ receptor allosteric modulators have promising in vitro and in vivo properties.

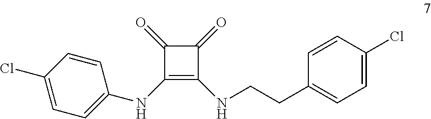 [SMILES: O=c1c(NCCc2ccc(Cl)cc2)c(Nc2ccc(Cl)cc2)c1=O]

Therefore, an alternate approach to target CB1 pathway by an allosteric modulator has emerged as a promising strategy to modulate this therapeutically valued CB1 receptor while avoiding side effects of orthosteric ligands.

The present disclosure describes the development of squaramide-based CB1 allosteric modulators. They are structurally distinct from previously disclosed urea-based CB1 allosteric modulators. Particularly, Compound 1 (FIG. 1) demonstrated better metabolic stability in rat liver microsomes than PSNCBAM-1/Known Compound 2 and was effective at attenuating cocaine reseeking behavior at a low dose of 5.6 mg/kg i.p.

In some embodiments, the presently disclosed subject matter provides a compound having a structure of Formula (I) or of Formula (II).

The presently disclosed compounds may comprise pharmaceutically acceptable salts. Such salts include, but are not limited to, pharmaceutically acceptable acid addition salts, pharmaceutically acceptable base addition salts, pharmaceutically acceptable metal salts, ammonium and alkylated ammonium salts, and combinations thereof.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N, N′-dibenzylethylenediamine, chloroprocaine, diethanolamine, procaine, N-benzylphenethylamine, diethylamine, piperazine, tris (hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like.

Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like.

Furthermore, the presently disclosed compounds may have one or more polymorph or amorphous crystalline forms, which, as such, are intended to be included in the scope of the presently disclosed subject matter. In addition, some of the compounds of the presently disclosed subject matter may form solvates with water (i.e., hydrates) or common organic solvents (e.g., tetrahydrofuran (THF), ethanol (EtOH), methanol (MeOH), etc.). Accordingly, solvates of the compounds of the present disclosure are also intended to be encompassed within the scope of the presently disclosed subject matter.

The compounds disclosed herein may be formulated in accordance with the routine procedures adapted for a desired administration route. Accordingly, in some embodiments, the presently disclosed subject matter provides a pharmaceutical composition comprising a therapeutically effective amount of a compound as disclosed hereinabove (e.g., a compound of the present disclosure, or a pharmaceutically acceptable salt or solvate thereof, and a pharmaceutically acceptable carrier. The therapeutically effective amount may be determined by testing the compounds in an in vitro or in vivo model and then extrapolating therefrom for dosages in subjects of interest, e.g., humans. The therapeutically effective amount should be enough to exert a therapeutically useful effect in the absence of undesirable side effects in the subject to be treated with the composition.

Pharmaceutically acceptable carriers are well known to those skilled in the art and include, but are not limited to, from about 0.01 to about 0.1M and preferably 0.05M phosphate buffer or 0.8% saline. Such pharmaceutically acceptable carriers may be aqueous or non-aqueous solutions, suspensions and emulsions. Examples of non-aqueous solvents suitable for use in the presently disclosed subject matter include, but are not limited to, propylene glycol, polyethylene glycol, vegetable oils such as olive oil, and injectable organic esters such as ethyl oleate. Aqueous carriers suitable for use in the presently disclosed subject matter include, but are not limited to, water, ethanol, alcoholic/aqueous solutions, glycerol, emulsions or suspensions, including saline and buffered media. Oral carriers may be elixirs, syrups, capsules, tablets and the like.

Liquid carriers suitable for use in the presently disclosed subject matter may be used in preparing solutions, suspensions, emulsions, syrups, elixirs and pressurized compounds. The active ingredient may be dissolved or suspended in a pharmaceutically acceptable liquid carrier such as water, an organic solvent, a mixture of both or pharmaceutically acceptable oils or fats. The liquid carrier may contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the presently disclosed subject matter include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier may also include an oily ester such as ethyl oleate and isopropyl myristate. Sterile liquid carriers are useful in sterile liquid form comprising compounds for parenteral administration. The liquid carrier for pressurized compounds disclosed herein may be halogenated hydrocarbon or other pharmaceutically acceptable propellent.

Solid carriers suitable for use in the presently disclosed subject matter include, but are not limited to, inert substances such as lactose, starch, glucose, methyl-cellulose, magnesium stearate, dicalcium phosphate, mannitol and the like. A solid carrier may further include one or more substances acting as flavoring agents, lubricants, solubilizers, suspending agents, fillers, glidants, compression aids, binders or tablet-disintegrating agents; it may also be an encapsulating material. In powders, the carrier may be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Parenteral carriers suitable for use in the presently disclosed subject matter include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives may also be present, such as, for example, antimicrobials, antioxidants, chelating agents, inert gases and the like.

Carriers suitable for use in the presently disclosed subject matter may be mixed as needed with disintegrants, diluents, granulating agents, lubricants, binders and the like using conventional techniques known in the art. The carriers may also be sterilized using methods that do not deleteriously react with the compounds, as is generally known in the art. The compounds disclosed herein may take such forms as suspensions, solutions or emulsions in oily or aqueous vehicles, and may contain formulatory agents such as suspending, stabilizing and/or dispersing agents. The compounds disclosed herein may also be formulated as a preparation for implantation or injection. Thus, for example, the compounds may be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient may be in powder form for constitution with a suitable vehicle, e.g., sterile pyrogen-free water, before use. Suitable formulations for each of these methods of administration may be found, for example, in Remington: The Science and Practice of Pharmacy, A. Gennaro, ed., 20th edition, Lippincott, Williams & Wilkins, Philadelphia, Pa.

For example, formulations for parenteral administration may contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or may be aqueous solutions containing, for example, polyoxyethylene-9-auryl ether, glycocholate and deoxycholate, or oily solutions for administration in the form of nasal drops, or as a gel to be applied intranasally. Formulations for parenteral administration may also include glycocholate for buccal administration, methoxysalicylate for rectal administration, or citric acid for vaginal administration.

Further, formulations for intravenous administration may comprise solutions in sterile isotonic aqueous buffer. Where necessary, the formulations may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it may be dispensed in a formulation with an infusion bottle containing sterile pharmaceutical grade water, saline or dextrose/water. Where the compound is administered by injection, an ampule of sterile water for injection or saline may be provided so that the ingredients may be mixed prior to administration.

Suitable formulations further include aqueous and non-aqueous sterile injection solutions that may contain antioxidants, buffers, bacteriostats, bactericidal antibiotics and solutes that render the formulation isotonic with the bodily fluids of the intended recipient; and aqueous and non-aqueous sterile suspensions, which may include suspending agents and thickening agents.

The compounds may further be formulated for topical administration. Suitable topical formulations include one or more compounds in the form of a liquid, lotion, cream or gel. Topical administration may be accomplished by application directly on the treatment area. For example, such application may be accomplished by rubbing the formulation (such as a lotion or gel) onto the skin of the treatment area, or by spray application of a liquid formulation onto the treatment area.

In some formulations, bioimplant materials may be coated with the compounds so as to improve interaction between cells and the implant.

Formulations of the compounds may contain minor amounts of wetting or emulsifying agents, or pH buffering agents. The formulations comprising the compound may be a liquid solution, suspension, emulsion, tablet, pill, capsule, sustained release formulation, or powder.

The compounds may be formulated as a suppository, with traditional binders and carriers such as triglycerides.

Oral formulations may include standard carriers such as pharmaceutical grades of mannitol, lactose, starch, magnesium stearate, polyvinyl pyrollidone, sodium saccharine, cellulose, magnesium carbonate, etc.

In some embodiments, the pharmaceutical composition comprising the compound of the presently disclosed subject matter may include an agent which controls release of the compound, thereby providing a timed or sustained release compound.

As described hereinabove, CB1 and CB2 cannabinoid receptors belong to the G protein-coupled receptor (GCPR) family, a receptor super-family with a distinctive pattern of seven transmembrane domains, which inhibits N-type calcium channels and/or adenylate cyclase to inhibit Q-type calcium channels.

CB1 receptors are present in the CNS, predominately expressed in brain regions associated with memory and movement such as the hippocampus (memory storage), cerebellum (coordination of motor function, posture and balance), basal ganglia (movement control), hypothalamus (thermal regulation, neuroendocrine release, appetite), spinal cord (nociception), cerebral cortex (emesis) and periphery regions such as lymphoid organs (cell mediated and innate immunity), vascular smooth muscle cells (blood pressure), gastrointestinal tract (innate antiinflammatory response in the gastrointestinal tract (e.g., in the esophagus, duodenum, jejunum, ileum and colon), controlling esophageal and gastrointestinal motility), lung smooth muscle cells (bronchodilation), eye ciliary body (intraocular pressure). CB2 receptors appear to be primarily expressed peripherally in lymphoid tissue (cell mediated and innate immunity), peripheral nerve terminals (peripheral nervous system), spleen immune cells (immune system modulation) and retina (intraocular pressure). CB2 mRNA is also found in the CNS in cerebellar granule cells (coordinating motor function),

Thus, cannabinoid receptor allosteric modulators, including the compounds of the present disclosure, are useful for treating, ameliorating or preventing a cannabinoid receptor mediated syndrome, disorder or disease including, but not limited to, controlling appetite, regulating metabolism, diabetes, glaucoma-associated intraocular pressure, pain, social and mood disorders, seizure-related disorders, substance abuse disorders, learning, cognition and/or memory disorders, bowel disorders, respiratory disorders, locomotor activity disorders, movement disorders, immune disorders or inflammation disorders, controlling organ contraction and muscle spasm, enhancing learning, cognition and/or memory, regulating cell growth (e.g., treating cancer), providing neuroprotection and the like.

Appetite related syndromes, disorders or diseases include obesity, overweight condition, anorexia, bulimia, cachexia, dysregulated appetite and the like. Obesity related syndromes, disorders or diseases include obesity as a result of genetics, diet, food intake volume, metabolic syndrome, disorder or disease, hypothalmic disorder or disease, age, reduced activity, abnormal adipose mass distribution, abnormal adipose compartment distribution and the like. Metabolism related syndromes, disorders or diseases include metabolic syndrome, dyslipidemia, elevated blood pressure, diabetes, insulin sensitivity or resistance, hyperinsulinemia, hypercholesterolemia, hyperlipidemias, hypertriglyceridemias, atherosclerosis, hepatomegaly, steatosis, abnormal alanine aminotransferase levels, inflammation, atherosclerosis and the like. Diabetes related syndromes, disorders or diseases include glucose dysregulation, insulin resistance, glucose intolerance, hyperinsulinemia, dyslipidemia, hypertension, obesity and the like.

Type II diabetes mellitus (non-insulin-dependent diabetes mellitus (NIDDM)) is a metabolic disorder (i.e., a metabolism related syndrome, disorder or disease) in which glucose dysregulation and insulin resistance results in chronic, long-term medical complications for both adolescents and adults affecting the eyes, kidneys, nerves and blood vessels and may lead to blindness, end-stage renal disease, myocardial infarction or limb amputation and the like. Glucose dysregulation includes the inability to make sufficient insulin (abnormal insulin secretion) and the inability to effectively use insulin (resistance to insulin action in target organs and tissues). Individuals suffering from Type II diabetes mellitus have a relative insulin deficiency. That is, in such individuals, plasma insulin levels are normal to high in absolute terms, although they are lower than predicted for the level of plasma glucose that is present. Type II diabetes mellitus is characterized by the following clinical signs or symptoms: persistently elevated plasma glucose concentration or hyperglycemia; polyuria; polydipsia and/or polyphagia; chronic microvascular complications such as retinopathy, nephropathy and neuropathy; and macrovascular complications such as hyperlipidemia and hypertension. These micro- and macro-vascular complications may lead to blindness, end-stage renal disease, limb amputation and myocardial infarction. Insulin Resistance Syndrome (IRS) (also referred to as Syndrome X, Metabolic Syndrome or Metabolic Syndrome X) is a disorder that presents risk factors for the development of Type II diabetes and cardiovascular disease including glucose intolerance, hyperinsulinemia, insulin resistance, dyslipidemia (e.g. high triglycerides, low HDL-cholesterol and the like), hypertension and obesity.

Social or mood related syndromes, disorders or diseases include depression, anxiety, psychosis, social affective disorders or cognitive disorders and the like. Substance abuse related syndromes, disorders or diseases include drug abuse, drug withdrawal, alcohol abuse, alcohol withdrawal, nicotine withdrawal, cocaine abuse, cocaine withdrawal, heroin abuse, heroin withdrawal and the like. Learning, cognition or memory related syndromes, disorders or diseases include memory loss or impairment as a result of age, disease, side effects of medications (adverse events) and the like.

Muscle spasm syndromes, disorders or diseases include multiple sclerosis, cerebral palsy and the like. Locomotor activity and movement syndromes, disorders or diseases include stroke, Parkinson's disease, multiple sclerosis, epilepsy and the like. Bowel related syndromes, disorders or diseases include bowel dysmotility associated disorders (either accompanied by pain, diarrhea or constipation or without), irritable bowel syndrome (and other forms of bowel dysmotility and the like), inflammatory bowel diseases (such as ulcerative colitis, Crohn's disease and the like) and celiac disease. Respiratory related syndromes, disorders or diseases include chronic pulmonary obstructive disorder, emphysema, asthma, bronchitis and the like. Immune or inflammation related syndromes, disorders or diseases include allergy, rheumatoid arthritis, dermatitis, autoimmune disease, immunodeficiency, chronic neuropathic pain and the like.

Cell growth related syndromes, disorders or diseases include cancer, such as, but not limited to endometrial cancer, hepatocellular cancer, ovarian cancer, breast cancer, pancreatic cancer, colorectal cancer, lung cancer, prostate cancer, and renal cell carcinoma, and the like. Pain related syndromes, disorders or diseases include central and peripheral pathway mediated pain, bone and joint pain, migraine headache associated pain, cancer pain, menstrual cramps, labor pain and the like. Neurodegenerative related syndromes, disorders or diseases include Parkinson's Disease, multiple sclerosis, epilepsy, ischemia or secondary biochemical injury collateral to traumatic head or brain injury, brain inflammation, eye injury or stroke and the like.

Based on the antagonistic activity, the presently disclosed compounds may be useful as agents for prevention and/or treatment of a CB1 receptor-mediated diseases such as psychosis including schizophrenia, anxiety disorders, stress, depression, epilepsy, neurodegenerative disorders, spinocerebellar disorders, cognitive disorders, craniocerebral trauma, panic attack, peripheral neuropathy, glaucoma, migraine, Parkinson's disease, Alzheimer's disease, Huntington's disease, Raynaud's syndrome, tremor, obsessive-compulsive disorders (OCD), amnesia, geriatric dementia, thymic disorders, Tourette's syndrome, tardive dyskinesia, bipolar disorders, cancer, drug-induced dyskinesia, dystonia, septic shock, hemorrhagic shock, hypotension, insomnia, immunological diseases including inflammations, multiple sclerosis, emesis, diarrhea, asthma, appetite disorders such as bulimarexia, anorexia and the like, obesity, non insulin-dependent diabetes mellitus (NIDDM), memory disorders, urinary disorders, cardiovascular disorders, infertility disorders, infections, demyelination-related diseases, neuroinflammation, viral encephalitis, cerebral vascular incidents, cirrhosis of the liver or gastrointestinal disorders including intestinal transit disorders. In addition, the presently disclosed compounds may be used as agents for the treatment of substance addiction. For example, in some embodiments, the presently disclosed compounds may be used to treat withdrawal from a chronic treatment, alcohol dependence or drug abuse (e.g., an opioid, barbiturate, marijuana, cocaine, heroin, amphetamine, phencyclidine, a hallucinogenic agent, a benzodiazepine compound and the like). Furthermore, the presently disclosed compounds may be useful as an agent for enhancing analgesic activity of analgesic or narcotic drugs and the like; or an agent for smoking cessation (withdrawal from smoking or nicotine dependence).

Accordingly, in some embodiments, the presently disclosed subject matter provides a method of treating a CB1R mediated disease or condition in a subject in need thereof, wherein the method comprises administering to the subject a therapeutically effective amount of a compound of the present disclosure or a pharmaceutically acceptable salt or solvate thereof or a pharmaceutical composition thereof.

With respect to the methods of the presently disclosed subject matter, a preferred subject is a vertebrate subject. A preferred vertebrate is warm-blooded; a preferred warm-blooded vertebrate is a mammal. The subject treated by the presently disclosed methods is desirably a human, although it is to be understood that the principles of the presently disclosed subject matter indicate effectiveness with respect to all vertebrate species which are to be included in the term “subject.” In this context, a vertebrate is understood to be any vertebrate species in which treatment of a CB1R-mediated condition is desirable. As used herein, the term “subject” includes both human and animal subjects. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, also provided is the treatment of livestock, including, but not limited to, domesticated swine, ruminants, ungulates, horses (including race horses), poultry, and the like. In some embodiments, the subject is a human.

In some embodiments, the CB1R-mediated disease or condition is selected from the group including, but not limited to, drug addiction (e.g., alcohol, tobacco or other substance addiction), obesity, cancer (e.g., endometrial cancer, hepatocellular cancer, ovarian cancer, breast cancer, pancreatic cancer, colorectal cancer, lung cancer, prostate cancer, renal cell carcinoma, or desmotrophic small round cell tumors), pain (e.g., chronic pain, acute pain, somatic pain, visceral pain, meropathic pain, inflammatory pain), female infertility, memory loss, cognitive dysfunction, Parkinson's disease, dyskinesia, tardive dyskinesia, Alzheimer's disease, amyotrophic lateral sclerosis (ALS), Tourette's Syndrome, stroke, atherosclerosis, hypotension, intestinal hypoactivity in paralytic ileus, inflammation, osteoporosis, hypercholesterolemia, hyslipidemia, diabetes, retinopathy, glaucoma, anxiety, depression and other mood disorders, gastrointestinal disorders, and metabolic disorders.

The treatment of anxiety, for example, may include the treatment of anxiety disorders, such as, but not limited to, generalized anxiety disorder (GAD), post-traumatic stress disorder (PTSD), obsessive-compulsive disorder (OCD), panic disorder, social phobia, agoraphobia, or other more particular phobias. Eating disorders include, but are not limited to, anorexia, bulimia, and binge eating. Mood disorders include, but are not limited to, manic depression (bipolar disorder), major depression, and post-partum depression. Cognitive dysfunction includes disorders such as, for example, dementia, Attention Deficit Hyperactivity Disorder (ADHD), autism and Autism Spectrum Disorders (ASD), Down's Syndrome, traumatic brain injury (TBI), dyslexia, and the like. Alcoholism and substance abuse-related disorders may include abuse and/or addiction to alcohol, nicotine, or other drugs (e.g., opiates (e.g., heroin), cannabinoids, inhalants and psychostimulants such as cocaine, amphetamine and methamphetamine).

More particularly, diseases or conditions wherein inhibition of biological activity at, or signalling via, the CB1R is desirable include, but are not limited to obesity, alcoholism, and other substance abuse and/or addiction-related disorders. Thus, in some embodiments, the presently disclosed subject matter provides a method of treating obesity in a subject in need thereof, wherein the method comprises administering to the subject a compound of the present disclosure or a pharmaceutically acceptable salt or solvate thereof or a pharmaceutical composition thereof. In some embodiments, the subject is a human.

By way of further example, the presently disclosed CB1R allosteric modulators may find application in the treatment of substance use, abuse and/or addiction (including drug, alcohol and nicotine addiction), addictive behavior and symptoms and conditions associated with substance abuse and addiction, as exemplified herein. In some embodiments, the addiction is to at least one of nicotine, ethanol, cocaine, opioids, amphetamines, marijuana, or a synthetic cannabinoid agonist.

Addiction to substances such as alcohol, opiates, cannabinoids, nicotine marijuana, and psychostimulants is typically associated with a number of adverse or negative behaviors exhibited by addicts, which behaviors may serve to exacerbate, prolong or induce relapse into use or abuse of the substance, reinforce or exacerbate the addiction, or induce relapse into addiction and addictive behavior patterns. Other examples of negative behaviors associated with substance use or addiction include anxiety, dysphoria, stress reactivity, and cue reactivity. One particular problem with alcoholism, as with substance addiction in general, is the chronic relapsing nature of the disorder. This behavior pattern may be effectively modelled in rodents, where numerous studies have demonstrated the ability of drug priming, psychological stress or the re-presentation of cues previously associated with drug availability to reinstate drug-seeking behavior following extinction, even in the absence of subsequent drug rewards.

In some embodiments, the presently disclosed subject matter provides a method for the prevention or inhibition of substance abuse and/or addiction, an addictive behavior, or of a symptom, behavior, or condition associated with substance abuse and/or addiction, the method comprising administering to a subject in need thereof an effective amount of a CB1R allosteric modulator compound as disclosed herein or a pharmaceutically acceptable salt or solvate thereof) or a pharmaceutically acceptable composition comprising such a compound. In some embodiments, the subject is a human.

In some embodiments, the behavior associated with substance abuse and/or addiction comprises substance use (i.e., self-administration) and/or substance seeking behavior. In some embodiments, the substance abuse and/or addiction comprises alcohol abuse and/or addiction (i.e., alcoholism). In some embodiments, the substance abuse and/or addiction comprises nicotine abuse and/or addiction. In some embodiments, the substance abuse and/or addiction comprises opiate abuse and/or addiction. In some embodiments, the behavior associated with substance abuse or addiction is relapse.

An effective amount of the compounds disclosed herein comprise amounts sufficient to produce a noticeable effect, such as, but not limited to, a reduction or cessation of self-administration of alcohol or another substance of abuse, weight loss, lack of weight gain, etc.). Actual dosage levels of active ingredients in a therapeutic compound of the presently disclosed subject matter may be varied so as to administer an amount of the active compound that is effective to achieve the desired therapeutic response for a particular subject and/or application. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

The therapeutically effective amount of a compound may depend on a number of factors. For example, the species, age, and weight of the subject, the precise condition requiring treatment and its severity, the nature of the formulation, and the route of administration are all factors that may be considered. In some embodiments, the therapeutically effective amount is in the range of about 0.1 to about 100 mg/kg body weight of the subject per day. In some embodiments, the therapeutically effective amount is in the range of from about 0.1 to about 20 mg/kg body weight per day. Thus, for a 70 kg adult mammal, one example of an actual amount per day would be between about 10 and about 2000 mg. This amount may be given in a single dose per day or in a number (e.g., 2, 3, 4, or 5) of sub-doses per day such that the total daily dose is the same. The effective amount of a salt or solvate thereof may be determined as a proportion of the effective amount of the compound per se.

A compound of the presently disclosed subject matter may also be useful as adjunctive, add-on or supplementary therapy for the treatment of the above-mentioned diseases/disorders. Said adjunctive, add-on or supplementary therapy means the concomitant or sequential administration of a compound of the presently disclosed subject matter to a subject who has already received administration of, who is receiving administration of, or who will receive administration of one or more additional therapeutic agents for the treatment of the indicated conditions, for example, one or more known anti-depressant, anti-psychotics or anxiolytic agents.

In some embodiments, the presently disclosed subject matter provides a compound of the present disclosure for use as an active therapeutic substance. In some embodiments, the compound is for use in the treatment of a disease mediated by CB1R. In some embodiments, the presently disclosed subject matter provides the use of the compound of the present disclosure for the preparation of a medicament for the treatment of a disease mediated by CB1R.

In some embodiments, the presently disclosed subject matter provides a method of modulating the activity of CB1R, wherein the method comprises contacting a sample comprising CB1R with a compound of one of the present disclosure, or a pharmaceutically acceptable salt or solvate thereof, or a pharmaceutical composition thereof. In some embodiments, the sample is an ex vivo sample. In some embodiments, the sample comprises a biological fluid, e.g. plasma, cerebrospinal fluid, saliva. In some embodiments, the sample comprises an organ, tissue, cell or cell extract. In some embodiments, the sample is from a subject. In some embodiments, the method may further comprises contacting the sample with second compound, such as a compound having or suspected of having CB1R agonist or antagonist activity.

The presently disclosed antagonists may be prepared using standard synthetic methodology known in the art. For example, the compounds may be made by the methods described hereinbelow or variations thereof that will be apparent to persons skilled in the art based on the present disclosure. As necessary, protecting groups known in the art may be utilized during the synthesis of the compounds.

### Synthetic Schemes and Examples

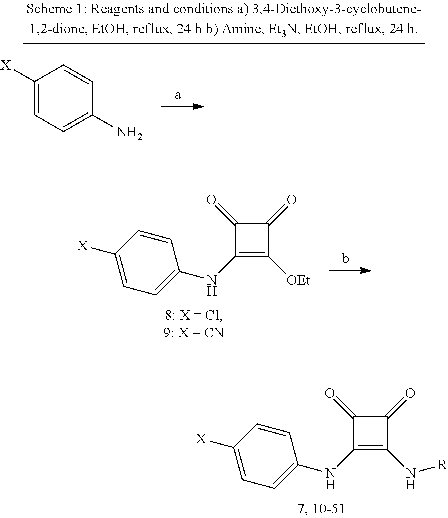

General procedure (GP): A solution of 2 (1 eq.), the corresponding amine (1.2 eq.) and triethylamine (1.1 eq.) in ethanol (0.15 M) was refluxed for 24 h. Upon cooling to room temperature, the precipitate was filtered and dried to afford the desired product.

3-[(4-Chlorophenyl)amino]-4-ethoxycyclobut-3-ene-1,2-dione (8): To a refluxing solution of 3,4-diethoxy-3-cyclobutene-1,2-dione (0.53 mL, 3.6 mmol) in ethanol was added an ethanol solution of 4-chloroaniline (0.38 g, 3 mmol) dropwise. The reaction was refluxed overnight. After cooling to room temperature, the reaction was filtered and the white solid precipitate was washed with ice-cold ethanol and collected as the desired product (0.39 g, 51%). ¹H NMR (300 MHz, CHCl₃) δ 7.32-7.37 (m, 2H), 7.27-7.30 (m, 2H), 4.89 (q, J=7.16 Hz, 2H), 1.52 (t, J=7.16 Hz, 3H). MS(ESI) [M+H]⁺ m/z=252.2.

3-[(4-Cyanophenyl)amino]-4-ethoxycyclobut-3-ene-1,2-dione (9): was prepared in a similar manner to 8 from 3,4-diethoxy-3-cyclobutene-1,2-dione (1.5 mL, 10.2 mmol) and 4-cyanoaniline (1 g, 8.5 mmol) as white solid (1.5 g, 75%). ¹H NMR (300 MHz, DMSO-d₆) δ 11.07 (s, 1H), 7.82 (d, J=8.67 Hz, 2H), 7.56 (d, J=8.85 Hz, 2H), 4.79 (q, J=6.97 Hz, 2H), 1.43 (t, J=7.06 Hz, 3H). MS(ESI) [M+H]⁺ m/z=243.2.

3-(Benzylamino)-4-[(4-chlorophenyl)amino]cyclobut-3-ene-1,2-dione (10) was prepared from 8 (0.08 g, 0.3 mmol) and benzylamine (0.04 mL, 0.36 mmol) according to GP as white solid (0.09 g, 90% yield). ¹H NMR (300 MHz, DMSO-d6) δ 9.73 (br. s., 1H), 8.02 (br. s., 1H), 7.28-7.49 (m, 9H), 4.81 (s, 2H). MS(ESI) [M+H]⁺ m/z=313.2.

3-[(4-Chlorophenyl)amino]-4-[(2-phenylethyl)amino]cyclobut-3-ene-1,2-dione (11) was prepared from 8 (0.08 g, 0.3 mmol) and phenethylamine (0.045 mL, 0.36 mmol) according to GP as white solid (0.09 g, 92% yield). ¹H NMR (300 MHz, DMSO-d6) δ 9.73 (br. s., 1H), 7.65 (br. s., 1H), 7.19-7.47 (m, 9H), 3.86 (d, J=5.46 Hz, 2H), 2.90 (t, J=5.84 Hz, 2H). MS(ESI) [M+H]⁺ m/z=327.2

3-[(4-Chlorophenyl)amino]-4-[(3-phenylpropyl)amino]cyclobut-3-ene-1,2-dione (12) was prepared from 8 (0.08 g, 0.3 mmol) and 3-phenylpropylamine (0.051 mL, 0.36 mmol) according to GP as white solid (0.10 g, 93% yield). ¹H NMR (300 MHz, DMSO-d6) δ 9.70 (br. s., 1H), 7.69 (br. s., 1H), 7.41 (q, J=8.92 Hz, 4H), 7.14-7.33 (m, 5H), 3.63 (q, J=5.97 Hz, 2H), 2.61-2.70 (m, 2H), 1.82-1.96 (m, 2H). MS(ESI) [M+H]⁺ m/z=341.2.

3-[(4-Chlorophenyl)amino]-4-{[2-(4-chlorophenyl)ethyl]amino}cyclobut-3-ene-1,2-dione (13) was prepared from 8 (0.08 g, 0.3 mmol) and 4-chlorophenethylamine (0.05 mL, 0.36 mmol) according to GP as white solid (0.09 g, 86% yield). ¹H NMR (300 MHz, DMSO-d6) δ 9.71 (br. s., 1H), 7.62 (br. s., 1H), 7.24-7.50 (m, 9H), 3.84 (d, J=4.52 Hz, 2H), 2.89 (t, J=5.18 Hz, 2H). MS(ESI) [M+H]⁺ m/z=361.2.

3-[(4-Chlorophenyl)amino]-4-{[2-(3-methylphenyl)ethyl]amino}cyclobut-3-ene-1,2-dione (14) was prepared from 8 (0.08 g, 0.3 mmol) and 3-methylphenethylamine (0.051 mL, 0.36 mmol) according to GP as white solid (0.09 g, 83% yield). ¹H NMR (300 MHz, DMSO-d6) δ 9.74 (br. s., 1H), 7.64 (br. s., 1H), 7.30-7.50 (m, 4H), 7.15-7.25 (m, 1H), 7.07 (d, J=8.85 Hz, 3H), 3.84 (d, J=5.09 Hz, 2H), 2.85 (t, J=6.50 Hz, 2H), 2.28 (s, 3H). MS(ESI) [M+H]⁺ m/z=341.2.

3-[(4-Chlorophenyl)amino]-4-{[2-(4-methylphenyl)ethyl]amino}cyclobut-3-ene-1,2-dione (15) was prepared from 8 (0.08 g, 0.3 mmol) and 4-methylphenethylamine (0.051 mL, 0.36 mmol) according to GP as white solid (0.09 g, 83% yield). ¹H NMR (300 MHz, DMSO-d6) δ 9.73 (br. s., 1H), 7.62 (br. s., 1H), 7.30-7.49 (m, 4H), 7.05-7.23 (m, 4H), 3.83 (d, J=6.03 Hz, 2H), 2.85 (t, J=6.59 Hz, 2H), 2.27 (s, 3H). MS(ESI) [M+H]⁺ m/z=341.2.

3-[(4-Chlorophenyl)amino]-4-{[2-(2-methoxyphenyl)ethyl]amino}cyclobut-3-ene-1,2-dione (16) was prepared from 8 (0.08 g, 0.3 mmol) and 4-methylphenethylamine (0.054 mL, 0.36 mmol) according to GP as white solid (0.08 g, 75% yield). ¹H NMR (300 MHz, DMSO-d6) δ 9.67 (br. s., 1H), 7.62 (br. s., 1H), 7.33-7.47 (m, 4H), 7.14-7.26 (m, 2H), 6.97 (d, J=8.10 Hz, 1H), 6.85-6.92 (m, 1H), 3.81 (d, J=5.27 Hz, 2H), 3.77 (s, 3H), 2.86 (t, J=6.78 Hz, 2H). MS(ESI) [M+H]⁺ m/z=357.2.

3-[(4-Chlorophenyl)amino]-4-{[2-(3-methoxyphenyl)ethyl]amino}cyclobut-3-ene-1,2-dione (17) was prepared from 8 (0.08 g, 0.3 mmol) and 3-methoxyphenethylamine (0.054 mL, 0.36 mmol) according to GP as white solid (0.08 g, 75% yield). ¹H NMR (300 MHz, DMSO-d6) δ 9.60 (br. s., 1H), 7.64 (br. s., 1H), 7.33-7.46 (m, 4H), 7.23 (t, J=7.91 Hz, 1H), 6.76-6.88 (m, 3H), 3.86 (t, J=6.12 Hz, 2H), 3.73 (s, 3H), 2.87 (t, J=6.88 Hz, 2H). MS(ESI) [M+H]⁺ m/z=357.2.

3-[(4-Chlorophenyl)amino]-4-{[2-(4-methoxyphenyl)ethyl]amino}cyclobut-3-ene-1,2-dione (18) was prepared from 8 (0.08 g, 0.3 mmol) and 3-methoxyphenethylamine (0.053 mL, 0.36 mmol) according to GP as white solid (0.08 g, 84% yield). ¹H NMR (300 MHz, DMSO-d6) δ 9.70 (br. s., 1H), 7.64 (br. s., 1H), 7.34-7.46 (m, 4H), 7.18 (d, J=8.10 Hz, 2H), 6.88 (d, J=7.72 Hz, 2H), 3.77-3.87 (m, 2H), 3.72 (s, 3H), 2.83 (t, J=6.50 Hz, 2H). MS(ESI) [M+H]⁺ m/z=357.2.

3-[(4-Chlorophenyl)amino]-4-({2-[2-(trifluoromethyl)phenyl]ethyl}amino)cyclobut-3-ene-1,2-dione (19) was prepared from 8 (0.08 g, 0.3 mmol) and 2-(trifluoromethyl)phenethylamine (0.053 mL, 0.36 mmol) according to GP as white solid (0.09 g, 76% yield). ¹H NMR (300 MHz, DMSO-d6) δ 9.71 (br. s., 1H), 7.67-7.78 (m, 2H), 7.65 (d, J=7.35 Hz, 1H), 7.52-7.59 (m, 1H), 7.45-7.50 (m, 1H), 7.34-7.42 (m, 4H), 3.88 (d, J=6.03 Hz, 2H), 3.08 (t, J=6.88 Hz, 2H). MS(ESI) [M+H]⁺ m/z=395.2.

3-[(4-Chlorophenyl)amino]-4-({2-[3-(trifluoromethyl)phenyl]ethyl}amino)cyclobut-3-ene-1,2-dione (20) was prepared from 8 (0.08 g, 0.3 mmol) and 3-(trifluoromethyl)phenethylamine (0.057 mL, 0.36 mmol) according to GP as white solid (0.095 g, 81% yield). ¹H NMR (300 MHz, DMSO-d6) δ 9.72 (br. s., 1H), 7.64 (s, 2H), 7.58 (br. s., 3H), 7.33-7.42 (m, 4H), 3.88 (d, J=5.46 Hz, 2H), 3.01 (t, J=6.88 Hz, 2H). MS(ESI) [M+H]⁺ m/z=395.2.

3-[(4-Chlorophenyl)amino]-4-({2-[4-(trifluoromethyl)phenyl]ethyl}amino)cyclobut-3-ene-1,2-dione (21) was prepared from 8 (0.08 g, 0.3 mmol) and 4-(trifluoromethyl)phenethylamine (0.057 mL, 0.36 mmol) according to GP as white solid (0.09 g, 76% yield). ¹H NMR (300 MHz, DMSO-d6) δ 9.73 (br. s., 1H), 7.69 (d, J=8.10 Hz, 2H), 7.64 (br. s., 1H), 7.51 (d, J=7.91 Hz, 2H), 7.33-7.44 (m, 4H), 3.89 (d, J=5.65 Hz, 2H), 3.00 (t, J=6.78 Hz, 2H). MS(ESI) [M+H]⁺ m/z=395.2.

3-[(4-Chlorophenyl)amino]-4-{[2-(4-fluorophenyl)ethyl]amino}cyclobut-3-ene-1,2-dione (22) was prepared from 8 (0.08 g, 0.3 mmol) and 4-fluorophenethylamine (0.057 mL, 0.36 mmol) according to GP as white solid (0.09 g, 87% yield). ¹H NMR (300 MHz, DMSO-d6) δ 9.67 (br. s., 1H), 7.63 (br. s., 1H), 7.35-7.45 (m, 4H), 7.30 (dd, J=5.75, 8.38 Hz, 2H), 7.10-7.19 (m, 2H), 3.83 (t, J=6.12 Hz, 2H), 2.89 (t, J=6.97 Hz, 2H). MS(ESI) [M+H]⁺ m/z=345.2.

3-[(4-Chlorophenyl)amino]-4-{[2-(2-chlorophenyl)ethyl]amino}cyclobut-3-ene-1,2-dione (23) was prepared from 8 (0.08 g, 0.3 mmol) and 2-chlorophenethylamine (0.05 mL, 0.36 mmol) according to GP as white solid (0.10 g, 93% yield). ¹H NMR (300 MHz, DMSO-d6) δ 9.49 (br. s., 1H), 7.69 (br. s., 1H), 7.23-7.54 (m, 8H), 3.74-4.04 (m, 2H), 2.90-3.13 (m, 2H). MS(ESI) [M+H]⁺ m/z=361.2.

3-[(4-Chlorophenyl)amino]-4-{[2-(3-chlorophenyl)ethyl]amino}cyclobut-3-ene-1,2-dione (7) was prepared from 8 (0.08 g, 0.3 mmol) and 3-chlorophenethylamine (0.05 mL, 0.36 mmol) according to GP as white solid (0.10 g, 93% yield). ¹H NMR (300 MHz, DMSO-d6) δ 9.72 (br. s., 1H), 7.63 (br. s., 1H), 7.27-7.45 (m, 7H), 7.23 (d, J=7.16 Hz, 1H), 3.86 (d, J=4.52 Hz, 2H), 2.91 (t, J=6.78 Hz, 2H). MS(ESI) [M+H]⁺ m/z=361.2.

3-[(4-Chlorophenyl)amino]-4-{[2-(2-fluorophenyl)ethyl]amino}cyclobut-3-ene-1,2-dione (24) was prepared from 8 (0.08 g, 0.3 mmol) and 2-fluorophenethylamine (0.047 mL, 0.36 mmol) according to GP as white solid (0.09 g, 90% yield). ¹H NMR (300 MHz, DMSO-d6) δ 9.70 (br. s., 1H), 7.66 (br. s., 1H), 7.24-7.45 (m, 6H), 7.11-7.21 (m, 2H), 3.85 (d, J=5.84 Hz, 2H), 2.94 (t, J=6.59 Hz, 2H). MS(ESI) [M+H]⁺ m/z=345.2.

3-[(4-Chlorophenyl)amino]-4-{[2-(3-fluorophenyl)ethyl]amino}cyclobut-3-ene-1,2-dione (25) was prepared from 8 (0.08 g, 0.3 mmol) and 3-fluorophenethylamine (0.047 mL, 0.36 mmol) according to GP as white solid (0.09 g, 90% yield). ¹H NMR (300 MHz, DMSO-d6) δ 9.69 (br. s., 1H), 7.64 (br. s., 1H), 7.34-7.43 (m, 5H), 7.07-7.17 (m, 2H), 7.01-7.07 (m, 1H), 3.80-3.92 (m, 2H), 2.92 (t, J=6.88 Hz, 2H). MS(ESI) [M+H]⁺ m/z=345.2.

3-[(4-Chlorophenyl)amino]-4-({2-[3-(dimethylamino)phenyl]ethyl}amino)cyclobut-3-ene-1,2-dione (26) was prepared from 8 (0.09 g, 0.35 mmol) and 3-(dimethylamino)phenethylamine (0.085 g, 0.42 mmol) according to GP as white solid (0.075 g, 58% yield). ¹H NMR (300 MHz, DMSO-d6) δ 9.79 (br. s., 1H), 7.64 (br. s., 1H), 7.36-7.45 (m, 4H), 7.07-7.16 (m, 1H), 6.48-6.71 (m, 3H), 3.85 (d, J=6.22 Hz, 2H), 2.86 (s, 6H), 2.78-2.85 (m, 2H). MS(ESI) [M+H]⁺ m/z=370.4.

3-[(4-Chlorophenyl)amino]-4-({2-[4-(dimethylamino)phenyl]ethyl}amino)cyclobut-3-ene-1,2-dione (27) was prepared from 8 (0.097 g, 0.38 mmol) and 4-(dimethylamino)phenethylamine (0.093 g, 0.46 mmol) according to GP as white solid (0.057 g, 40% yield). ¹H NMR (300 MHz, DMSO-d6) δ 9.75 (br. s., 1H), 7.61 (br. s., 1H), 7.33-7.46 (m, 4H), 7.07 (d, J=8.48 Hz, 2H), 6.69 (d, J=8.29 Hz, 2H), 3.79 (d, J=5.84 Hz, 2H), 2.85 (s, 5H), 2.77 (t, J=6.78 Hz, 2H). MS(ESI) [M+H]⁺ m/z=370.4.

4-[(2-{[2-(3-Chlorophenyl)ethyl]amino}-3,4-dioxocyclobut-1-en-1-yl)amino]benzonitrile (28) was prepared from 9 (0.072 g, 0.3 mmol) and 3-chlorophenethylamine (0.05 mL, 0.36 mmol) according to GP as white solid (0.084 g, 80% yield). ¹H NMR (300 MHz, DMSO-d6) δ 10.01 (br. s., 1H), 7.77 (d, J=8.67 Hz, 3H), 7.55 (d, J=8.48 Hz, 2H), 7.27-7.40 (m, 3H), 7.24 (d, J=6.97 Hz, 1H), 3.81-3.93 (m, J=6.03 Hz, 2H), 2.92 (t, J=6.88 Hz, 2H). MS(ESI) [M+H]⁺ m/z=352.2.

3-(Biphenyl-3-ylamino)-4-[(4-chlorophenyl)amino]cyclobut-3-ene-1,2-dione (29) was prepared from 8 (0.053 g, 0.21 mmol) and 3-aminobiphenyl (0.043 g, 0.25 mmol) according to GP as white solid (0.06 g, 75% yield). ¹H NMR (300 MHz, DMSO-d6) δ 7.85 (s, 1H), 7.70 (d, J=7.54 Hz, 2H), 7.33-7.54 (m, 12H). MS(ESI) [M+H]⁺ m/z=375.2.

3-{3-[6-(Pyrrolidin-1-yl)pyridin-2-yl]-phenyl}-4-[(4-chlorophenyl)amino]cyclobut-3-ene-1,2-dione (30) was prepared from 8 (0.073 g, 0.29 mmol) and 3-[6-(pyrrolidin-1-yl)pyridin-2-yl]aniline (0.084 g, 0.35 mmol) according to GP as white solid (0.105 g, 81% yield). ¹H NMR (300 MHz, DMSO-d6) δ 8.20 (s, 1H), 7.75 (d, J=6.78 Hz, 1H), 7.40-7.61 (m, 7H), 7.13 (d, J=7.35 Hz, 1H), 6.43 (d, J=8.48 Hz, 1H), 1.96 (t, J=6.40 Hz, 4H). MS(ESI) [M+H]⁺ m/z=455.2.

3-[(4-Chlorophenyl)amino]-4-{[3-(1,3-thiazol-4-yl)phenyl]amino}cyclobut-3-ene-1,2-dione (31) was prepared from 8 (0.049 g, 0.19 mmol) and 3-(1,3-thiazol-4-yl)aniline (0.034 g, 0.19 mmol) according to GP as white solid (0.105 g, 72% yield). ¹H NMR (300 MHz, DMSO-d6) δ 9.92-10.09 (m, 2H), 9.22 (d, J=1.70 Hz, 1H), 8.03-8.17 (m, 2H), 7.71 (d, J=7.35 Hz, 1H), 7.38-7.56 (m, 7H). MS(ESI) [M+H]⁺ m/z=382.2.

3-[(4-Chlorophenyl)amino]-4-{[3-(thiophen-3-yl)phenyl]amino}cyclobut-3-ene-1,2-dione (32) was prepared from 8 (0.05 g, 0.2 mmol) and 3-(thiophen-3-yl)aniline (0.034 g, 0.2 mmol) according to GP as white solid (0.06 g, 80% yield). ¹H NMR (300 MHz, DMSO-d6) δ 9.99 (d, J=4.90 Hz, 2H), 7.86-7.93 (m, 2H), 7.68 (dd, J=3.01, 4.90 Hz, 1H), 7.56 (d, J=4.90 Hz, 1H), 7.49-7.54 (m, 2H), 7.40-7.48 (m, 4H), 7.31 (d, J=7.35 Hz, 1H). MS(ESI) [M+H]⁺ m/z=381.2.

3-[(4-Chlorophenyl)amino]-4-[(4′-fluorobiphenyl-3-yl)amino]cyclobut-3-ene-1,2-dione (33) was prepared from 8 (0.07 g, 0.28 mmol) and 4′-fluorobiphenyl-3-amine (0.05 g, 0.28 mmol) according to GP as white solid (0.07 g, 73% yield). ¹H NMR (300 MHz, DMSO-d6) δ 9.99 (s, 2H), 7.83 (s, 1H), 7.69-7.78 (m, 2H), 7.42-7.53 (m, 5H), 7.28-7.41 (m, 4H). MS(ESI) [M−H]⁻ m/z=391.2.

3-(Biphenyl-4-ylamino)-4-[(4-chlorophenyl)amino]cyclobut-3-ene-1,2-dione (34) was prepared from 8 (0.04 g, 0.16 mmol) and biphenyl-4-amine (0.03 g, 0.16 mmol) according to GP as white solid (0.024 g, 40% yield). ¹H NMR (300 MHz, DMSO-d6) δ 10.03 (br. s., 2H), 7.65-7.73 (m, 4H), 7.58 (d, J=8.67 Hz, 2H), 7.42-7.54 (m, 6H), 7.31-7.38 (m, 1H). MS(ESI) [M−H]⁻ m/z=373.2.

3-[(4-Chlorophenyl)amino]-4-{[2-(2-methylphenyl)ethyl]amino}cyclobut-3-ene-1,2-dione (35) was prepared from 8 (0.05 g, 0.20 mmol) and 2-methylphenethylamine hydrochloride (0.029 g, 0.20 mmol) according to GP as white solid (0.030 g, 44% yield). ¹H NMR (400 MHz, DMSO-d6) δ 9.72 (br. s., 1H), 7.65 (br. s., 1H), 7.30-7.41 (m, 4H), 7.04-7.18 (m, 4H), 3.73-3.81 (m, 1H), 2.85 (t, J=7.18 Hz, 2H), 2.27 (s, 3H). MS(ESI) [M+H]⁺ m/z=341.0.

3-[(4-Chlorophenyl)amino]-4-{[2-(pyridin-2-yl)ethyl]amino}cyclobut-3-ene-1,2-dione (36) was prepared from 8 (0.05 g, 0.20 mmol) and 2-(2-pyridyl)ethylamine (0.029 g, 0.20 mmol) according to GP as white solid (0.060 g, 92% yield). ¹H NMR (400 MHz, DMSO-d₆) δ 9.72 (br. s., 1H), 8.49 (d, J=4.13 Hz, 1H), 7.67-7.74 (m, 1H), 7.65 (br. s., 1H), 7.26-7.40 (m, 5H), 7.19-7.24 (m, 1H), 3.93-4.02 (m, 1H), 3.04 (t, J=6.72 Hz, 2H). MS(ESI) [M+H]⁺ m/z=328.0.

3-[(4-Chlorophenyl)amino]-4-{[2-(pyridin-4-yl)ethyl]amino}cyclobut-3-ene-1,2-dione (37) was prepared from 8 (0.05 g, 0.20 mmol) and 2-(4-pyridyl)ethylamine (0.029 g, 0.20 mmol) according to GP as white solid (0.060 g, 92% yield). ¹H NMR (400 MHz, DMSO-d₆) δ 9.69 (br. s., 1H), 8.46 (d, J=5.20 Hz, 2H), 7.59 (br. s., 1H), 7.29-7.44 (m, 4H), 7.27 (d, J=5.35 Hz, 2H), 3.86 (m, 2H), 2.89 (t, J=6.80 Hz, 2H). MS(ESI) [M+H]⁺ m/z=328.0.

3-[(4-Chlorophenyl)amino]-4-{[2-(4-bromophenyl)ethyl]amino}cyclobut-3-ene-1,2-dione (38) was prepared from 8 (0.05 g, 0.20 mmol) and 4-bromophenethylamine hydrochloride (0.048 g, 0.20 mmol) according to GP as white solid (0.062 g, 77% yield). ¹H NMR (400 MHz, DMSO-d₆) δ 9.69 (br. s., 1H), 7.57 (br. s., 1H), 7.48 (d, J=7.95 Hz, 2H), 7.30-7.40 (m, 3H), 7.20 (d, J=8.10 Hz, 2H), 3.80 (d, J=6.42 Hz, 2H), 2.83 (t, J=6.80 Hz, 2H). MS(ESI) [M+H]⁺ m/z=405.0.

3-[(4-Chlorophenyl)amino]-4-{[2-(thiophen-2-yl)ethyl]amino}cyclobut-3-ene-1,2-dione (39) was prepared from 8 (0.05 g, 0.20 mmol) and 2-(2-aminoethyl)thiophene (0.030 g, 0.20 mmol) according to GP as white solid (0.058 g, 88% yield). ¹H NMR (400 MHz, DMSO-d₆) δ 9.75 (br. s., 1H), 7.66 (br. s., 1H), 7.31-7.41 (m, 5H), 6.93-6.97 (m, 1H), 6.89-6.92 (m, 1H), 3.83 (m, 2H), 3.08 (t, J=6.65 Hz, 2H). MS(ESI) [M+H]⁺ m/z=333.0.

3-{[2-(A-acetylphenyl)ethyl]amino}-4-[(4-chlorophenyl)amino]cyclobut-3-ene-1,2-dione (40) was prepared from 8 (0.05 g, 0.20 mmol) and 1-[4-(2-aminoethyl)phenyl]ethanone hydrochloride (0.048 g, 0.20 mmol) according to GP as white solid (0.060 g, 82% yield). ¹H NMR (400 MHz, DMSO-d₆) δ 9.72 (br. s., 1H), 7.88 (d, J=7.64 Hz, 2H), 7.63 (br. s., 1H), 7.30-7.42 (m, 6H), 3.85 (d, J=5.96 Hz, 2H), 2.95 (t, J=6.72 Hz, 2H), 2.52 (s, 3H). MS(ESI) [M+H]⁺ m/z=369.0.

3-[(4-Chlorophenyl)amino]-4-{[2-(3,4-dichlorophenyl)ethyl]amino}cyclobut-3-ene-1,2-dione (41) was prepared from 8 (0.05 g, 0.20 mmol) and 3,4-dichlorophenethylamine (0.045 g, 0.20 mmol) according to GP as white solid (0.060 g, 76% yield). ¹H NMR (400 MHz, DMSO-d₆) δ 9.68 (br. s., 1H), 7.54 (br. s., 3H), 7.30-7.40 (m, 4H), 7.23 (d, J=7.95 Hz, 1H), 3.81 (m, 2H), 2.87 (t, J=6.57 Hz, 2H). MS(ESI) [M−H]⁻ m/z=395.0.

3-[(4-Chlorophenyl)amino]-4-{[2-(3,4-dimethylphenyl)ethyl]amino}cyclobut-3-ene-1,2-dione (42) was prepared from 8 (0.05 g, 0.20 mmol) and 3,4-dimethylphenethylamine (0.036 g, 0.20 mmol) according to GP as white solid (0.064 g, 91% yield). ¹H NMR (400 MHz, DMSO-d₆) δ 9.68 (br. s., 1H), 7.57 (br. s., 1H), 7.29-7.41 (m, 4H), 6.98-7.08 (m, 2H), 6.93 (d, J=7.03 Hz, 1H), 3.78 (br. s., 2H), 2.77 (t, J=6.88 Hz, 2H), 2.14 (d, J=3.67 Hz, 6H). MS(ESI) [M+H]⁺ m/z=355.0.

3-[(4-Chlorophenyl)amino]-4-{[2-(3-hydroxyphenyl)ethyl]amino}cyclobut-3-ene-1,2-dione (43) was prepared from 8 (0.05 g, 0.20 mmol) and 3-hydroxyphenethylamine hydrochloride (0.041 g, 0.20 mmol) according to GP as white solid (0.065 g, 95% yield). ¹H NMR (400 MHz, DMSO-d₆) δ 9.72 (br. s., 1H), 9.30 (s, 1H), 7.59 (br. s., 1H), 7.30-7.40 (m, 4H), 7.07 (t, J=7.64 Hz, 1H), 6.61-6.67 (m, 2H), 6.58 (d, J=8.10 Hz, 1H), 3.78 (d, J=6.11 Hz, 2H), 2.76 (t, J=6.88 Hz, 2H). MS(ESI) [M−H]⁻ m/z=342.0.

3-[(4-Chlorophenyl)amino]-4-{[2-(2,4-dichlorophenyl)ethyl]amino}cyclobut-3-ene-1,2-dione (44) was prepared from 8 (0.05 g, 0.20 mmol) and 2,4-dichlorophenethylamine (0.045 g, 0.20 mmol) according to GP as white solid (0.065 g, 83% yield). ¹H NMR (400 MHz, DMSO-d₆) δ 9.67 (br. s., 1H), 7.55-7.65 (m, 2H), 7.30-7.40 (m, 6H), 3.81 (d, J=6.11 Hz, 2H), 2.98 (t, J=6.57 Hz, 2H). MS(ESI) [M−H]⁻ m/z=395.0

3-[(4-Chlorophenyl)amino]-4-{[2-(piperidin-1-yl)ethyl]amino}cyclobut-3-ene-1,2-dione (45) was prepared from 8 (0.05 g, 0.20 mmol) and 1-(2-aminoethyl)piperidine (0.031 g, 0.20 mmol) according to GP as white solid (0.040 g, 60% yield). ¹H NMR (400 MHz, DMSO-d₆) δ 9.83 (br. s., 1H), 7.56 (br. s., 1H), 7.38-7.44 (m, 2H), 7.31-7.37 (m, 2H), 3.67 (br. s., 2H), 2.42 (t, J=5.96 Hz, 2H), 2.34 (br. s., 4H), 1.41-1.50 (m, 4H), 1.35 (d, J=4.74 Hz, 2H). MS(ESI) [M+H]⁺ m/z=334.0.

3-[(4-Chlorophenyl)amino]-4-({2-[4-(4-chlorophenyl)piperazin-1-yl]ethyl}amino)cyclobut-3-ene-1,2-dione (46) was prepared from 8 (0.05 g, 0.20 mmol) and 1-(4-chlorophenyl)piperazine (0.047 g, 0.20 mmol) according to GP as white solid (0.077 g, 96% yield). ¹H NMR (400 MHz, DMSO-d₆) δ 9.62 (s, 1H), 7.31-7.36 (m, 2H), 7.17-7.26 (m, 4H), 6.98 (d, J=9.02 Hz, 2H), 3.85 (br. s., 4H), 3.23-3.29 (m, 4H). MS(ESI) [M−H]⁻ m/z=401.0.

3-[(4-Chlorophenyl)amino]-4-{[2-(2,4-difluorophenyl)ethyl]amino}cyclobut-3-ene-1,2-dione (47) was prepared from 8 (0.05 g, 0.20 mmol) and 2,4-difluorophenethylamine (0.045 g, 0.20 mmol) according to GP as white solid (0.030 g, 42% yield). ¹H NMR (400 MHz, DMSO-d₆) δ 9.69 (br. s., 1H), 7.59 (br. s., 1H), 7.24-7.44 (m, 7H), 7.07 (br. s., 1H), 3.74-3.87 (m, 2H), 2.81-2.90 (m, 2H). MS(ESI) [M+H]⁺ m/z=363.0.

3-[(4-Chlorophenyl)amino]-4-({2-[4-(diethylamino)phenyl]ethyl}amino)cyclobut-3-ene-1,2-dione (48) was prepared from 8 (0.05 g, 0.20 mmol) and 4-(N,N-diethylamino)phenethylamine (0.045 g, 0.20 mmol) according to GP as white solid (0.034 g, 43% yield). ¹H NMR (400 MHz, DMSO-d₆) δ 9.73 (br. s., 1H), 7.58 (br. s., 1H), 7.30-7.38 (m, 4H), 7.00 (d, J=7.64 Hz, 2H), 6.57 (d, J=7.95 Hz, 2H), 3.74 (d, J=6.72 Hz, 2H), 3.19-3.26 (m, 4H), 2.70 (t, J=6.80 Hz, 2H), 1.01 (t, J=6.88 Hz, 6H). MS(ESI) [M+H]⁺ m/z=398.2.

3-[(4-Chlorophenyl)amino]-4-{[2-(4-piperidin-1-ylphenyl)ethyl]amino}cyclobut-3-ene-1,2-dione (49) was prepared from 8 (0.154 g, 0.61 mmol) and 2-(4-piperidin-1-ylphenyl)ethylamine (0.15 g, 0.73 mmol) according to GP as white solid (0.05 g, 19% yield). ¹H NMR (300 MHz, DMSO-d₆) δ 9.74 (br. s., 1H), 7.61 (br. s., 1H), 7.32-7.41 (m, 4H), 7.08 (d, J=8.48 Hz, 2H), 6.87 (d, J=8.29 Hz, 2H), 3.79 (d, J=6.03 Hz, 2H), 3.07 (br. s., 4H), 2.78 (t, J=6.69 Hz, 2H), 1.60 (br. s., 4H), 1.51 (d, J=4.52 Hz, 2H). MS(ESI) [M+H]⁺ m/z=409.0.

3-[(4-Chlorophenyl)amino]-4-{[2-(4-pyrrolidin-1-ylphenyl)ethyl]amino}cyclobut-3-ene-1,2-dione (50) was prepared from 8 (0.20 g, 0.8 mmol) and 2-(4-pyrrolidin-1-ylphenyl)ethyl]amine (0.23 g, 0.10 mmol) according to GP as white solid (0.25 g, 79% yield). ¹H NMR (300 MHz, DMSO-d₆) δ 9.68-9.80 (m, 1H), 7.54-7.65 (m, 1H), 7.31-7.45 (m, 4H), 7.05 (d, J=7.54 Hz, 2H), 6.49 (d, J=7.35 Hz, 2H), 3.77 (br. s., 2H), 3.18 (br. s., 4H), 2.74 (d, J=6.40 Hz, 2H), 1.93 (br. s., 4H). MS(ESI) [M+H]⁺ m/z=396.0.

3-{[2-(4-Azetidin-1-ylphenyl)ethyl]amino}-4-[(4-chlorophenyl)amino]cyclobut-3-ene-1,2-dione (51) was prepared from 8 (0.21 g, 0.82 mmol) and 2-(4-azetidin-1-ylphenyl)ethyl]amine (0.17 g, 0.98 mmol) according to GP as white solid (0.30 g, 96% yield). ¹H NMR (300 MHz, DMSO-d₆) δ 9.78 (br. s., 1H), 7.65 (br. s., 1H), 7.34-7.46 (m, 4H), 7.05 (d, J=8.48 Hz, 2H), 6.36 (d, J=7.72 Hz, 2H), 3.68-3.84 (m, 6H), 2.76 (t, J=7.16 Hz, 2H), 2.21-2.33 (m, 2H). ¹H NMR (300 MHz, DMSO-d₆) δ 9.78 (br. s., 1H), 7.65 (br. s., 1H), 7.34-7.45 (m, 5H), 7.05 (d, J=8.48 Hz, 2H), 6.36 (d, J=7.72 Hz, 2H), 3.70-3.82 (m, 6H), 2.76 (t, J=7.16 Hz, 2H), 2.21-2.33 (m, 2H). MS(ESI) [M+H]⁺ m/z=382.0.

### In Vitro Assay Examples

Calcium Mobilization Assay: CHO-RD-HGA16 cells (Molecular Devices, San Jose, California, United States of America) stably expressing the human CB₁ receptor were plated into 96-well black-walled assay plates at 25,000 cells/well in 100 μL of Ham's F12 (supplemented with 10% fetal bovine serum, 100 units of penicillin/streptomycin, and 100 μg/mL Normocin) and incubated overnight at 37° C., 5% CO₂. Calcium 5 dye (Molecular Devices, San Jose, California, United States of America) was reconstituted according to the manufacturer's instructions. The reconstituted dye was diluted 1:40 in prewarmed (37° C.) assay buffer (lx HBSS, 20 mM HEPES, 2.5 mM probenecid, pH 7.4 at 37° C.). Growth medium was removed, and the cells were gently washed with 100 μL of prewarmed (37° C.) assay buffer. The cells were incubated for 45 min at 37° C., 5% CO₂ in 200 μL of the diluted Calcium 5 dye solution. For antagonist assays to determine IC₅₀ values, the EC₅₀ concentration of CP55,940 was prepared at 10× the desired final concentration in 0.25% BSA/0.5% DMSO/0.5% EtOH/assay buffer, aliquoted into 96-well polypropylene plates, and warmed to 37° C. Serial dilutions of the test compounds were prepared at 10× the desired final concentration in 2.25% BSA/4.5% DMSO/4.5% EtOH/assay buffer. After the dye loading incubation period, the cells were pretreated with 25 μL of the test compound serial dilutions and incubated for 15 min at 37° C. After the pretreatment incubation period, the plate was read with a FLIPR Tetra (Molecular Devices, San Jose, California, United States of America). Calcium-mediated changes in fluorescence were monitored every 1 s over a 90 s time period, with the Tetra adding 25 μL of the CP55,940 EC₅₀ concentration at the 10 s time point (excitation/emission: 485/525 nm). Relative fluorescence units (RFU) were plotted against the log of compound concentrations. For agonist screens, the above procedure was followed except that cells were pretreated with 2.25% BSA/4.5% DMSO/4.5% EtOH/assay buffer and the Tetra added single concentration dilutions of the test compounds prepared at 10× the desired final concentration in 0.25% BSA/0.5% DMSO/0.5% EtOH/assay buffer. Test compound RFUs were compared to the CP55,940 Eₘₐₓ RFUs to generate % Eₘₐₓ values. For the CB₂ agonist and antagonist assays, the same procedures were followed except that stable human CB₂—CHO-RD-HGA16 cells were used.

[³⁵S]GTPγS Binding Assay: For receptor signaling, membranes (10 μg protein) from either ICR mouse cerebellum mice (6-8 weeks old; Enviga International, Indianapolis, Indiana, United States of America) or HEK cells stably expressing CB1 receptor were preincubated in assay buffer for 10 min with 3 units/ml adenosine deaminase then incubated for 60 min at 30° C. with 30 μM GDP and 0.1 nM [³⁵S]GTPγS (Perkin Elmer Life Sciences, Boston, Massachusetts, United States of America). Non-specific binding was determined by adding 30 μM unlabeled GTPγS. Concentration response curves for allosteric modulators were conducted in the presence of CP55,940 (100 nM or 1 μM) to calculate IC₅₀ values.

cAMP Assay: The cAMP assay was performed as previously described. See Cawston et al. J. Med. Chem. 2015, 58, 5979-5988. Briefly, forskolin (FSK)-stimulated cyclic adenosine monophosphate (cAMP) production was measured in real-time using a transfected bioluminescence resonance energy transfer (BRET) cAMP sensor. The plasmid encodes a cAMP binding domain (Epac1) flanked by yellow fluorescent protein (YFP) and Renilla Luciferase (RLuc) assay, the latter of which may oxidize coelenterazine H and produce a photon as a byproduct. When cAMP is bound to the Epac1 domain, it separates RLuc and YFP so only Rluc emits a photon at a wavelength of 460 nm. When cAMP is not bound, RLuc may excite YFP, emitting light at wavelength 535 nm. A plate reader measures both wavelengths and their ratio, 460/535, is calculated to quantify cAMP levels where increases in the ratio indicate increases in cAMP. Human Embryonic Kidney 293 (HEK293) cells stably transfected with the human cannabinoid type-1 (CB₁) were maintained at 37° C. at 5% CO₂ and seeded in 100 mM dishes for transfection. The next day, cells were given fresh growth media and transfected with 5 μg of pcDNA3L-His-CAMYEL using linear polyethyleneimine (25 kDa, Polysciences, Warrington, Pennsylvania, United States of America) in 1:6 DNA:PEI (ATCC, Manassas, Virginia, United States of America) ratio. The next day, cells were lifted using 1 mM EDTA in PBS and spun down at 200×g for 5 min. The supernatant was removed, and cells were resuspended in growth media and plated on poly-D-lysine (Sigma Aldrich, St. Louis, Missouri, United States of America) coated white 96 well plates at 60,000 cells per well, filling 2 columns of 8 wells each per plate, i.e. 8 samples in duplicate per plate (Perkin Elmer, Waltham, Massachusetts, United States of America). The following day, media was removed, cells were rinsed with PBS and buffers/reagents/drugs added as following: At 0 min, 175 μL of stimulation buffer (5 mg/ml bovine serum albumin in HBSS including Ca²⁺ and Mg²⁺); at 10 min, 25 μL of allosteric modulators added; at 15 min: 25 μL coelenterazine added (5 μM final); at 25 min, 25 μL of forskolin (10 μM final) with or without CP55,940 (100 nM final) added. Immediately following addition of forskolin and the probe agonist CP55,940, the luminescence is measured at 460 nm and 535 nm simultaneously for 1 s per well for 22 min at 37° C. using a Clariostar (BMG Labtech, Ortenberg, Germany). Ratio of 460/535 is calculated for each time point and plotted across time and area under the curve analysis is conducted for each replicate and averaged by condition/day where each day serves as an independent experiment. Data were calculated as % FSK using the formula [(sample−basal)/(forskolin−basal)×100]. IC₅₀ values were calculated from these normalized concentration-response data using Prism 6 (Graphpad Software, San Diego, California, United States of America) using 3 parameter non-linear regression. Data are plotted as mean of at least N=3 independent experiments either normalized to forskolin (concentration response data) or the calculated 460/535 BRET ratio (time course data).

Data Analysis: For calcium mobilization experiments, data were fit to a three-parameter logistic curve to generate IC₅₀ values (GraphPad Prism 6.0, Graphpad Software, San Diego, California, United States of America). For [³⁵S]GTPγS experiments, data were normalized to maximal CP55,940 (100 nM) stimulation in the absence of test compound (i.e., vehicle=100%). Curve fits were accomplished using GraphPad Prism 6.0 (Graphpad Software, San Diego, California, United States of America) and data were fit to three-parameter nonlinear regression, with bottom and top constrained to >0 and =100, respectively, for IC₅₀ calculation.

Results: The presently disclosed compounds were characterized in the calcium mobilization assay using CHO cells overexpressing human CB1R and the [³⁵S]GTPγS binding assay in HEK cells overexpressing human CB1R as described previously.

TABLE 1
Allosteric Modulating Activities of arylalkylsquaramide derivatives
in the human CB₁ Calcium Mobilization and mouse CB₁ [³⁵S]GTPγS Binding Assays.

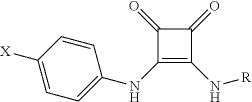

| | | | | | Human |
|---|---|---|---|---|---|
| | | | | | CB₁ |
| | | | | | HEK293 |
| | | | | Mouse CB₁ | membrane |
| | | | Human | [³⁵S]GTPγS | [³⁵S]GTPγS |
| | | | CB₁ | binding | binding |
| | | | Calcium | assay | assay |
| | | | assay | pIC50 ± | pIC50 ± |
| Compound | X///(R¹)ₘ | R///(CH₂)ₙ-Ring A-(R²)ₒ | IC₅₀ (nM)^{a} | SEM^{a} | SEM^{a} |
| 10 | Cl | 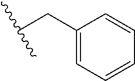 [SMILES: *Cc1ccccc1] | >10,000 | ND | ND |
| 11 | Cl | 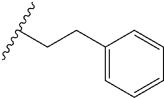 [SMILES: *CCc1ccccc1] | 210 ± 39 | ND | 6.11 ± 0.03 |
| 12 | Cl | 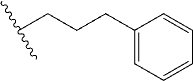 [SMILES: *CCCc1ccccc1] | 1037 ± 359 | ND | ND |
| 13 | Cl | 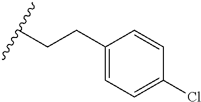 [SMILES: *CCc1ccc(Cl)cc1] | 222 ± 35 | 6.56 ± 0.10 | 6.37 ± 0.08 |
| 14 | Cl | 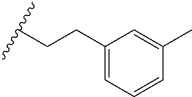 [SMILES: *CCc1cccc(C)c1] | 205 ± 41 | 6.78 ± 0.12 | 6.29 ± 0.11 |
| 15 | Cl | 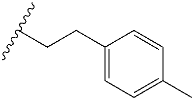 [SMILES: *CCc1ccc(C)cc1] | 164 ± 30 | 7.09 ± 0.27 | 5.93 ± 0.04 |
| 16 | Cl | 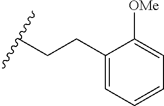 [SMILES: *CCc1ccccc1OC] | 1107 ± 88 | ND | <5 |
| 17 | Cl | 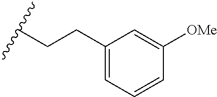 [SMILES: *CCc1cccc(OC)c1] | 350 ± 39 | ND | <5 |
| 18 | Cl | 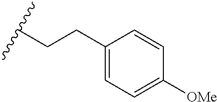 [SMILES: *CCc1ccc(OC)cc1] | 734 ± 310 | ND | <5 |
| 19 | Cl | 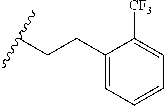 [SMILES: *CCc1ccccc1C(F)(F)F] | 933 + 131 | ND | <5 |
| 20 | Cl | 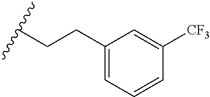 [SMILES: *CCc1cccc(C(F)(F)F)c1] | 360 ± 65 | ND | 6.04 ± 0.12 |
| 21 | Cl | 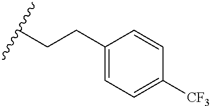 [SMILES: *CCc1ccc(C(F)(F)F)cc1] | 275 ± 13 | ND | 6.44 ± 0.11 |
| 22 | Cl | 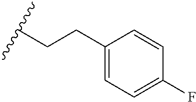 [SMILES: *CCc1ccc(F)cc1] | 337 ± 21 | ND | 6.07 ± 0.18 |
| 23 | Cl | 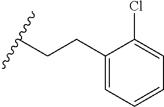 [SMILES: *CCc1ccccc1Cl] | 1288 ± 201 | ND | ND |
| 7 | Cl | 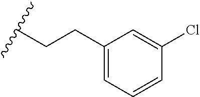 [SMILES: *CCc1cccc(Cl)c1] | 130 ± 12 | 6.64 ± 0.08 | 6.37 ± 0.10 |
| 24 | Cl | 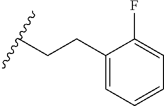 [SMILES: *CCc1ccccc1F] | 274 ± 33 | 6.18 ± 0.23 | 6.17 ± 0.28 |
| 25 | Cl | 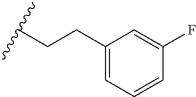 [SMILES: *CCc1cccc(F)c1] | 286 ± 28 | 6.62 ± 0.10 | 6.23 ± 0.25 |
| 26 | Cl | 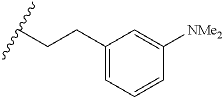 [SMILES: *CCc1cccc(N(C)C)c1] | 2104 ± 272 | 5.96 ± 0.12 | ND |
| 27 | Cl | 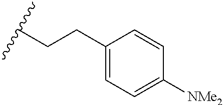 [SMILES: *CCc1ccc(N(C)C)cc1] | 535 ± 135 | 7.39 ± 0.10 | 6.17 ± 0.12 |
| 28 | CN | 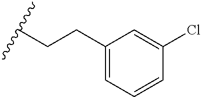 [SMILES: *CCc1cccc(Cl)c1] | 460 ± 66 | ND | 6.40 ± 0.17 |
| 35 | Cl | 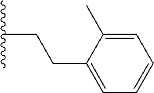 [SMILES: *CCc1ccccc1C] | 353 ± 37 | 5.57 ± 0.09 | 5.73 ± 0.15 |
| 36 | Cl | 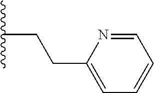 [SMILES: *CCc1ccccn1] | 958 ± 60 | 5.75 ± 0.17 | 5.79 ± 0.14 |
| 37 | Cl | 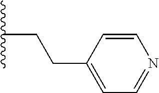 [SMILES: *CCc1ccncc1] | >10,000 | ND | ND |
| 38 | Cl | 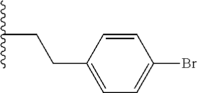 [SMILES: *CCc1ccc(Br)cc1] | 333 ± 7 | 6.06 ± 0.02 | 6.41 ± 0.07 |
| 39 | Cl | 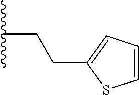 [SMILES: *CCc1cccs1] | 399 ± 22 | 5.87 ± 0.14 | 6.05 ± 0.11 |
| 40 | Cl | 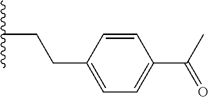 [SMILES: *CCc1ccc(C(C)=O)cc1] | 350 ± 10 | 6.70 ± 0.09 | 6.77 ± 0.04 |
| 41 | Cl | 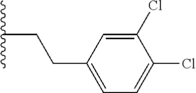 [SMILES: *CCc1ccc(Cl)c(Cl)c1] | 877 ± 31 | 6.53 ± 0.02 | 5.97 ± 0.34 |
| 42 | Cl | 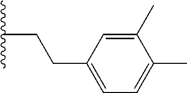 [SMILES: *CCc1ccc(C)c(C)c1] | 408 ± 66 | 6.48 ± 0.08 | 6.61 ± 0.23 |
| 43 | Cl | 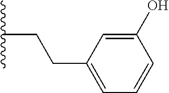 [SMILES: *CCc1cccc(O)c1] | 2071 ± 204 | 5.32 ± 0.14 | 5.00 ± 0.12 |
| 44 | Cl | 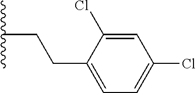 [SMILES: *CCc1ccc(Cl)cc1Cl] | 1135 ± 183 | 6.28 ± 0.05 | 6.48 ± 0.20 |
| 45 | Cl | 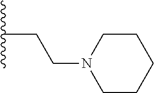 [SMILES: *CCN1CCCCC1] | >10,000 | ND | ND |
| 46 | Cl | 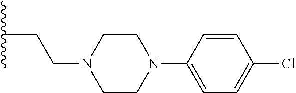 [SMILES: *CCN1CCN(c2ccc(Cl)cc2)CC1] | >10,000 | ND | ND |
| 47 | Cl | 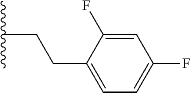 [SMILES: *CCc1ccc(F)cc1F] | 362 ± 30 | 6.61 ± 0.19 | 6.84 ± 0.08 |
| 48 | Cl | 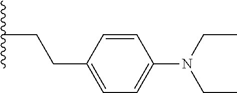 [SMILES: *CCc1ccc(N(CC)CC)cc1] | 639 ± 247 | 6.33 ± 0.14 | 6.11 ± 0.05 |
| 49 | Cl | 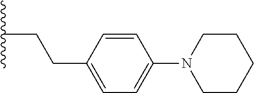 [SMILES: *CCc1ccc(N2CCCCC2)cc1] | >10,000 | ND | ND |
| 50 | Cl | 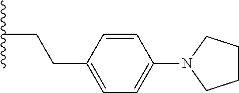 [SMILES: *CCc1ccc(N2CCCC2)cc1] | >10,000 | ND | ND |
| 51 | Cl | 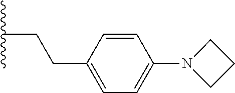 [SMILES: *CCc1ccc(N2CCC2)cc1] | >10,000 | ND | ND |

^{a}Values are the mean ± SEM of at least three independent experiments in duplicate.
ND Not determined

TABLE 2
Allosteric Modulating Activities of diarylsquaramide derivatives in
the human CB₁ Calcium Mobilization and mouse CB₁ [³⁵S]GTPγS Binding Assays.

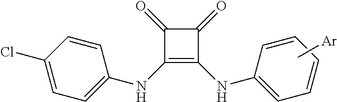

| | | | | Human CB₁ |
|---|---|---|---|---|
| | | | | HEK293 |
| | | | Mouse CB₁ | membrane |
| | | Human CB₁ | [³⁵S]GTPyS binding | [³⁵S]GTPγS |
| | | Calcium assay | assay | binding assay |
| Compound | Ar | IC₅₀ (nM)^{a} | pIC50 ± SEM^{a} | pIC50 ± SEM^{a} |
| 29 | 3-Ph | 408 ± 94 | ND | 6.61 ± 0.20 |
| 30 (GAT358) see WO 2015/27160 comparator | 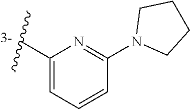 [SMILES: *c1cccc(N2CCCC2)n1] | 378 ± 124 | 6.02 ± 0.13 | <5 |
| 31 | 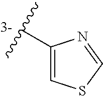 [SMILES: *c1cscn1] | 154 ± 14 | ND | ND |
| 32 | 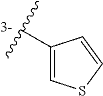 [SMILES: *c1ccsc1] | 463 ± 56 | ND | 6.02 ± 0.221 |
| 33 | 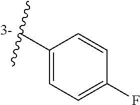 [SMILES: *c1ccc(F)cc1] | 575 ± 100 | 6.96 ± 0.14 | 6.30 ± 0.209 |
| 34 | 4-Ph | 1480 ± 297 | ND | ND |

^{a}Values are the mean ± SEM of at least three independent experiments in duplicate.
ND Not determined

### Stability, Solubility, Permeability and Pharmacokinetic Studies

Metabolic stability assessment: Compounds were incubated with rat liver microsomes at 37° C. for a total of 45 minutes. The reaction was performed at pH 7.4 in 100 mM potassium phosphate buffer containing 0.5 mg/mL of rat liver microsomal protein. Phase I metabolism was assessed by adding NADPH to a final concentration of 1 mM and collecting samples at time points 0, 5, 15, 30 and 45 minutes. All collected samples were quenched 1:1 with ice-cold stop solution (1 μM labetalol and 1 μM glyburide in acetonitrile) and centrifuged to remove precipitated protein. Resulting supernatants were further diluted 1:4 with acetonitrile:water (1:1). Samples were analyzed by LC/MS/MS and calculations for half-life, and in-vitro clearance were accomplished using Microsoft Excel (2007).

Kinetic solubility assessment: A 10 μL of test compound stock solution (20 mM DMSO) was combined with 490 μL of phosphate buffer solution to reach a targeted concentration of 400 μM. The solution was agitated on a VX-2500 multi-tube vortexer (VWR International, Radnor, Pennsylvania, United States of America) for 2 hours at room temperature. Following agitation, the sample was filtrated on a glass-fiber filter (1 μm) and the eluate was diluted 400-fold with a mixture of acetonitrile: water (1:1). On each experimental occasion, nicardipine and imipramine were assessed as reference compounds for low and high solubility, respectively. All samples were assessed in triplicate and analyzed by LC-MS/MS using electrospray ionization against standards prepared in the same matrix.

Results: In an effort to advance CB1R allosteric modulators for therapeutics development, preliminary ADME assessment of some of the presently disclosed compounds was performed.

TABLE 3
ADME data of squaramide analogs

| | | Rat | | | | | |
|---|---|---|---|---|---|---|---|
| | Rat | Liver | | | | | |
| | Liver | micro- | Ki- | | Pₐₚₚ | Pₐₚₚ | |
| | micro- | some | netic | % | A | B | Efflux |
| | some | Clearance | solu- | Plasma | to B | to A | ratio |
| Compound | T_{1/2} | (ul/min/ | bility | Protein | (x10⁻⁶ | (x10⁻⁶ | BA/ |
| Structure | (min) | mg) | (μM) | Bound | cm/s) | cm/s) | AB |
| 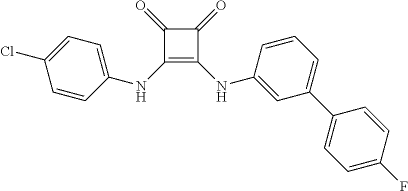 [SMILES: O=c1c(Nc2ccc(Cl)cc2)c(Nc2cccc(-c3ccc(F)cc3)c2)c1=O] | 23.7 | 59.6 | <1 | 99 | | | |
| 33 | | | | | | | |
| 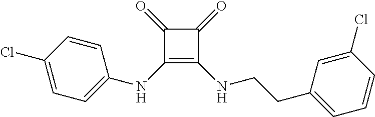 [SMILES: O=c1c(NCCc2cccc(Cl)c2)c(Nc2ccc(Cl)cc2)c1=O] | 40 | 34.7 | <1 | >99.74% | 3.1 | 6.4 | 2.1 |
| 7 | | | | | | | |

Permeability Assessment. Bidirectional MDCK-MDR1 permeability assay was performed by Paraza Pharma Inc. (Montreal, Canada). MDCK-mdr1 cells at passage 5 were seeded onto permeable polycarbonate supports in 12-well Costar Transwell plates and allowed to grow and differentiate for 3 days. On day 3, culture medium (DMEM supplemented with 10% FBS) was removed from both sides of the transwell inserts and cells were rinsed with warm HBSS. After the rinse step, the chambers were filled with warm transport buffer (HBSS containing 10 mM HEPES, 0.25% BSA, pH 7.4) and the plates were incubated at 37° C. for 30 min prior to TEER (Trans Epithelial Electric Resistance) measurements.

The buffer in the donor chamber (apical side for A-to-B assay, basolateral side for B-to-A assay) was removed and replaced with the working solution (10 μM test article in transport buffer). The plates were then placed at 37° C. under light agitation. At designated time points (30, 60 and 90 min), an aliquot of transport buffer from the receiver chamber was removed and replenished with fresh transport buffer. Samples were quenched with ice-cold ACN containing internal standard and then centrifuged to pellet protein. Resulting supernatants are further diluted with 50/50 ACN/H₂O (H₂O only for Atenolol) and submitted for LC-MS/MS analysis. Reported apparent permeability (Papp) values were calculated from single determination. Atenolol and propranolol were tested as low and moderate permeability references. Bidirectional transport of digoxin was assessed to demonstrate Pgp activity/expression.

The apparent permeability (Papp, measured in cm/s) of a compound is determined according to the following formula from two independent experiments in duplicate:

$$Papp = \frac{( dQ ) / ( dt )}{A * Ci * 60} ,$$

where dQ/dt is the net rate of appearance in the receiver compartment; A is the area of the Transwell measured in cm² (1.12 cm²); Ci is the initial concentration of compound added to the donor chamber; and 60 is the conversion factor for minute to second.

Pharmacokinetic Assessment. An in vivo pharmacokinetic assay was performed by Paraza Pharma Inc. (Montreal, Canada). On the morning of the PK study, male Sprague-Dawley rats weighing 258-277 g were dosed with either vehicle (5% Cremorphor, 5% ethanol in saline) or 7 (5.6 mg/kg, i.p.). At selected timepoints (0.25, 0.5, 1, 3, 5, 8 and 24 hours post dose), 2 rats were anesthetized with isoflurane gas to perform a cardiac puncture to collect blood (for plasma analysis), followed by whole body perfusion with phosphate saline buffer (PBS, pH 7.4) to wash out any remaining blood from the organs. Brains were harvested and homogenized by mechanical sheering with a polytron with 1:4 (w/v) 25% isopropanol in water. Brain homogenates were extracted for drug quantification by LC-MS/MS.

### Reinstatement of Extinguished Cocaine-Seeking Behavior

Adult male Sprague-Dawley rats (Harlan, Indianapolis, Indiana, United States of America) weighing 280-300 g were used in the study. Animals were housed individually on a 12/12 hr light/dark cycle (behavioral experiments were conducted during the light period) with free access to water and food except during experimental sessions.

The reinstatement procedure has been previously described. See Jing et al., Drug Alcohol Depend. 2014, 143, 251-256; and Thorn et al., Neuropsychopharmacology 2014, 39, 2309-2316. Briefly, rats were surgically implanted with a chronic indwelling jugular catheter. After one-week recovery, rats were trained to press the active lever (left lever) for infusion of cocaine (0.75 mg/kg/inf) under a fixed ratio [FR] schedule (starting FR=1, which was increased to FR 5 within 5 training sessions) schedule during daily 2-hr sessions for 14 days. Reinforcer deliveries were accompanied by the presentation of a stimulus light over the active lever followed by a 30-s time-out period during which lever presses had no programmed consequence. Following acquisition of cocaine self-administration, extinction of drug-seeking behavior took place during 2-hr daily sessions in which lever pressing produced no consequence. All other conditions remained unchanged. After 7 days of extinction, all rats reached the extinction criteria (total responses less than 20% of the training sessions).

Data analyses: Data are expressed as mean±S.E.M. Differences in active lever responding between the last extinction session and reinstatement session were determined with paired t tests (within subjects comparison). The effects of compound 7 on reinstatement were analyzed by a one-way analysis of variance (ANOVA) followed by post hoc Bonferroni's test (between subjects comparison). The effects of compound 7 on reinstatement was analyzed by Student's t test. P<0.05 was considered statistically significant.

Results: As shown in FIG. 1A, Compound 7 at 5.6 mg/kg (i.p.) was effective to attenuate cocaine-reseeking behavior in rats. Further, as shown in FIG. 1B, Compound 6 did not affect locomotion at 5.6 mg/kg (i.p.).

It will be understood that various details of the presently disclosed subject matter may be changed without departing from the scope of the presently disclosed subject matter. Furthermore, the foregoing description is for the purpose of illustration only, and not for the purpose of limitation.

All publications, patents, and patent applications cited in this specification are incorporated herein by reference for the teaching to which such citation is used.

Test compounds for the experiments described herein were employed in free or salt form.

The specific responses observed may vary according to and depending on the particular active compound selected or whether there are present carriers, as well as the type of formulation and mode of administration employed, and such expected variations or differences in the results are contemplated in accordance with practice of the present disclosure.

Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be obvious to those skilled in the art, and all modifications that do not depart from the spirit of the disclosure are intended to be included with the scope of the appended claims.

That which is claimed is:

1. A compound of formula (I): 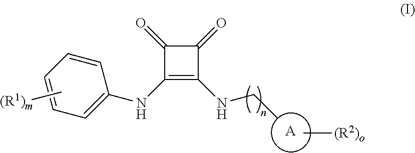
wherein
each R¹ is independently C₁-C₆ alkyl, C₂-C₆ alkenyl, C₂-C₆ alkynyl, halogen, C₁-C₆ haloalkyl, NO₂, CN, O—(C₁-C₆ alkyl), or N(R³)₂;
each R² is independently C₁-C₆ alkyl, C₂-C₆ alkenyl, C₂-C₆ alkynyl, halogen, C₁-C₆ haloalkyl, CN, NO₂, OH, O—(C₁-C₆ alkyl), O-(5- to 13-membered cycloalkyl), N(R³)₂, C(O)OH, C(O)C₁-C₆ alkyl, (C₁-C₆-alkyl)ₓ-(5- to 13-membered aryl)-(R⁴)ₚ, (C₁-C₆-alkyl)ₓ-(5- to 13-membered heterocyclyl containing one, two, or three heteroatoms selected from N, O, or S)—(R⁴)ₚ, (C₁-C₆-alkyl)ₓ-(5- to 13-membered heteroaryl containing one, two, or three heteroatoms selected from N, O, or S)—(R⁴)_{P};
each R³ is independently H or C₁₋₆ alkyl; or two R³ groups and the nitrogen atom to which they are attached form a 5-7 atom heterocyclic ring which may contain one or two more additional heteroatoms selected from N, O, and S;
each R⁴ is independently H, halo, C₁₋₆ alkyl, or N(R⁵)₂ wherein each R⁵ is independently H or C₁₋₆ alkyl;
each x independently is 0 or 1;
Ring A is selected from (i) C₃₋₆ cycloalkyl, (ii) C₃₋₆ heterocyclyl having one, two, or three heteroatoms each selected from N, O, or S, (iii) C₆ aryl, or (iv) C₅-6 heteroaryl having one, two, or three heteroatoms each selected form N, O, or S;
n is 0, 1, 2, 3, or 4;
m is 0, 1, 2, or 3;
o is 0, 1, 2, or 3; and
p is 0, 1, 2, or 3,
or a pharmaceutically acceptable salt or solvate thereof.

2. The compound of claim 1, wherein Ring A is C₆ aryl.

3. The compound of claim 2, wherein
each R¹ is independently halogen, C₁-C₄ alkyl, C₁-C₄ haloalkyl, CN, O—(C₁-C₆ alkyl), or N(R²)₂;
each R² is independently halogen, C₁-C₄ alkyl, C₁-C₄ haloalkyl, CN, NO₂, O—(C₁-C₆ alkyl), O-(5- to 13-membered cycloalkyl), N(R³)₂, C₁-C₈-alkyl)ₓ-(5- to 13-membered aryl), or (C₁-C₈-alkyl)ₓ-(5- to 13-membered heteroaryl), wherein each of aryl and heteroaryl is optionally substituted with one or more R⁴ groups; and
wherein the heteroaryl contains one, two, or three heteroatoms selected from N, O, and S;
each R³ is independently H or C₁₋₆ alkyl; or two R³ groups and the nitrogen atom to which they are attached form a 5-7 atom heterocyclic ring which may contain one or two more additional heteroatoms selected from N. O, and S;
each R⁴ is independently H, halo, C₁₋₆ alkyl or N(R¹)₂ wherein each R⁵ is independently H or C₁₋₆ alkyl;
n is 0, 1, 2, 3, or 4;
m is 1, 2, or 3;
o is 0, 1, 2, or 3,
or a pharmaceutically acceptable salt or solvate thereof.

4. The compound of claim 2, having formula (II): 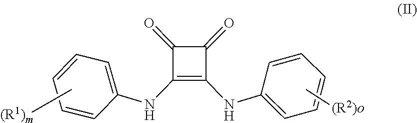
wherein
each R¹ is independently C₁-C₆ alkyl, C₂-C₆ alkenyl, C₂-C₆ alkynyl, halogen, C₁-C₆ haloalkyl, NO₂, CN, O—(C₁-C₆ alkyl), or N(R³)₂;
each R² is independently C₁-C₆ alkyl, C₂-C₆ alkenyl, C₂-C₆ alkynyl, halogen, C₁-C₆ haloalkyl, CN, O—(C₁-C₆ alkyl), N(R³)₂, C₁-C₆-alkyl)ₓ-(5- to 13-membered aryl), or (C₁-C₈-alkyl)ₓ-(5- to 13-membered heteroaryl), wherein each of aryl and heteroaryl is optionally substituted with one or more R⁴ groups, and wherein the heteroaryl contains one, two, or three heteroatoms selected from N, O, and S;
each R³ is independently H or C₁₋₆ alkyl; or two R³ groups and the nitrogen atom to which they are attached form a 5-7 atom heterocyclic ring which may contain one or two more additional heteroatoms selected from N. O, and S;
each R⁴ is independently H, halo, C₁₋₆ alkyl or N(R⁵)₂ wherein each R⁵ is independently H or C₁₋₆ alkyl;
m is 0, 1, 2, or 3; and
o is 0, 1, 2, or 3;
or a pharmaceutically acceptable salt or solvate thereof.

5. The compound of any one of claims 1 to 4, further comprising when R² is other than pyridyl, two R⁵ groups and the nitrogen atom to which they are attached may form a 5-7 membered hetero ring which may contain one or two more additional heteroatoms selected from N, O, and S, and which may contain one or more degrees of unsaturation.

6. The compound of claim 1, wherein Ring A is pyridinyl, thiophenyl, piperidinyl, or piperazinyl.

7. The compound of any one of claims 1 to 6, wherein R¹ is halogen or CN.

8. The compound of claim 7, wherein R¹ is halogen.

9. The compound of claim 8, wherein R¹ is Cl.

10. The compound of any one of claims 1 to 3 or 5 to 9, wherein n is 2.

11. The compound of any one of claims 1 to 10, wherein o is 0.

12. The compound of any one of claims 1 to 10, wherein o is at least 1 and each R² is selected from the group consisting of C₁₋₆alkyl, halogen, O(C₁₋₆ alkyl), C₁₋₆ haloalkyl, N(C₁₋₆ alkyl)₂, C(O)C₁₋₆ alkyl, OH, unsubstituted phenyl, phenyl substituted with halogen, unsubstituted pyridine, pyridine substituted with pyrroline, unsubstituted thiazole, and unsubstituted thiophene.

13. The compound of claim 12, wherein
o is 1 or 2, and
each R² is selected from the group consisting of C₁₋₆alkyl, halogen, O(C₁₋₆ alkyl), N(C₁₋₆ alkyl)₂, C(O)C₁₋₆ alkyl, unsubstituted phenyl, and phenyl substituted with halogen.

14. The compound of any one of claims 1 to 13, wherein R³ is CH₃.

15. The compound of any one of claims 1 to 14, wherein R⁴ is H, halo, or N(R⁵)₂.

16. The compound of any one of claims 1 to 15, wherein x is 0.

17. A compound selected from:
3-(Benzylamino)-4-[(4-chlorophenyl)amino]cyclobut-3-ene-1,2-dione (9);
3-[(4-Chlorophenyl)amino]-4-[(2-phenylethyl)amino]cyclobut-3-ene-1,2-dione (10);
3-[(4-Chlorophenyl)amino]-4-[(3-phenylpropyl)amino]cyclobut-3-ene-1,2-dione (11);
3-[(4-Chlorophenyl)amino]-4-{[2-(4-chlorophenyl)ethyl]amino}cyclobut-3-ene-1,2-dione (12);
3-[(4-Chlorophenyl)amino]-4-{[2-(3-methylphenyl)ethyl]amino}cyclobut-3-ene-1,2-dione (13);
3-[(4-Chlorophenyl)amino]-4-{[2-(4-methylphenyl)ethyl]amino}cyclobut-3-ene-1,2-dione (14);
3-[(4-Chlorophenyl)amino]-4-{[2-(2-methoxyphenyl)ethyl]amino}cyclobut-3-ene-1,2-dione (15);
3-[(4-Chlorophenyl)amino]-4-{[2-(3-methoxyphenyl)ethyl]amino}cyclobut-3-ene-1,2-dione (16);
3-[(4-Chlorophenyl)amino]-4-{[2-(4-methoxyphenyl)ethyl]amino}cyclobut-3-ene-1,2-dione (17);
3-[(4-Chlorophenyl)amino]-4-({2-[2-(trifluoromethyl)phenyl]ethyl}amino)cyclobut-3-ene-1,2-dione (18);
3-[(4-Chlorophenyl)amino]-4-({2-[3-(trifluoromethyl)phenyl]ethyl}amino)cyclobut-3-ene-1,2-dione (19);
3-[(4-Chlorophenyl)amino]-4-({2-[4-(trifluoromethyl)phenyl]ethyl}amino)cyclobut-3-ene-1,2-dione (20);
3-[(4-Chlorophenyl)amino]-4-{[2-(4-fluorophenyl)ethyl]amino}cyclobut-3-ene-1,2-dione (21);
3-[(4-Chlorophenyl)amino]-4-{[2-(2-chlorophenyl)ethyl]amino}cyclobut-3-ene-1,2-dione (22);
3-[(4-Chlorophenyl)amino]-4-{[2-(3-chlorophenyl)ethyl]amino}cyclobut-3-ene-1,2-dione (6);
3-[(4-Chlorophenyl)amino]-4-{[2-(2-fluorophenyl)ethyl]amino}cyclobut-3-ene-1,2-dione (23);
3-[(4-Chlorophenyl)amino]-4-{[2-(3-fluorophenyl)ethyl]amino}cyclobut-3-ene-1,2-dione (24);
3-[(4-Chlorophenyl)amino]-4-({2-[3-(dimethylamino)phenyl]ethyl}amino)cyclobut-3-ene-1,2-dione (25);
3-[(4-Chlorophenyl)amino]-4-({2-[4-(dimethylamino)phenyl]ethyl}amino)cyclobut-3-ene-1,2-dione (26);
4-[(2-{[2-(3-Chlorophenyl)ethyl]amino}-3,4-dioxocyclobut-1-en-1-yl)amino]benzonitrile (27);
3-(Biphenyl-3-ylamino)-4-[(4-chlorophenyl)amino]cyclobut-3-ene-1,2-dione (28);
3-{3-[6-(Pyrrolidin-1-yl)pyridin-2-yl]-phenyl}-4-[(4-chlorophenyl)amino]cyclobut-3-ene-1,2-dione (29);
3-[(4-Chlorophenyl)amino]-4-{[3-(thiophen-3-yl)phenyl]amino}cyclobut-3-ene-1,2-dione (31);
3-[(4-Chlorophenyl)amino]-4-[(4′-fluorobiphenyl-3-yl)amino]cyclobut-3-ene-1,2-dione (32);
3-(Biphenyl-4-ylamino)-4-[(4-chlorophenyl)amino]cyclobut-3-ene-1,2-dione (33);
3-[(4-Chlorophenyl)amino]-4-{[2-(2-methylphenyl)ethyl]amino}cyclobut-3-ene-1,2-dione;
3-[(4-Chlorophenyl)amino]-4-{[2-(pyridin-2-yl)ethyl]amino}cyclobut-3-ene-1,2-dione;
3-[(4-Chlorophenyl)amino]-4-{[2-(pyridin-4-yl)ethyl]amino}cyclobut-3-ene-1,2-dione;
3-[(4-Chlorophenyl)amino]-4-{[2-(4-bromophenyl)ethyl]amino}cyclobut-3-ene-1,2-dione;
3-[(4-Chlorophenyl)amino]-4-{[2-(thiophen-2-yl)ethyl]amino}cyclobut-3-ene-1,2-dione;
3-{[2-(A-acetylphenyl)ethyl]amino}-4-[(4-chlorophenyl)amino]cyclobut-3-ene-1,2-dione;
3-[(4-Chlorophenyl)amino]-4-{[2-(3,4-dichlorophenyl)ethyl]amino}cyclobut-3-ene-1,2-dione;
3-[(4-Chlorophenyl)amino]-4-{[2-(3,4-dimethylphenyl)ethyl]amino}cyclobut-3-ene-1,2-dione;
3-[(4-Chlorophenyl)amino]-4-{[2-(3-hydroxyphenyl)ethyl]amino}cyclobut-3-ene-1,2-dione;
3-[(4-Chlorophenyl)amino]-4-{[2-(2,4-dichlorophenyl)ethyl]amino}cyclobut-3-ene-1,2-dione; 3-[(4-Chlorophenyl)amino]-4-{[2-(piperidin-1-yl)ethyl]amino}cyclobut-3-ene-1,2-dione;
3-[(4-Chlorophenyl)amino]-4-({2-[4-(4-chlorophenyl)piperazin-1-yl]ethyl}amino)cyclobut-3-ene-1,2-dione;
3-[(4-Chlorophenyl)amino]-4-{[2-(2,4-difluorophenyl)ethyl]amino}cyclobut-3-ene-1,2-dione; and
3-[(4-Chlorophenyl)amino]-4-({2-[4-(diethylamino)phenyl]ethyl}amino)cyclobut-3-ene-1,2-dione,
or a pharmaceutically acceptable salt thereof.

18. A compound selected from
3-[(4-Chlorophenyl)amino]-4-{[2-(3-chlorophenyl)ethyl]amino}cyclobut-3-ene-1,2-dione (6);
3-[(4-Chlorophenyl)amino]-4-{[2-(4-chlorophenyl)ethyl]amino}cyclobut-3-ene-1,2-dione (12);
3-[(4-Chlorophenyl)amino]-4-{[2-(3-methylphenyl)ethyl]amino}cyclobut-3-ene-1,2-dione (13); and
3-[(4-Chlorophenyl)amino]-4-{[2-(4-methylphenyl)ethyl]amino}cyclobut-3-ene-1,2-dione (14),
or a pharmaceutically acceptable salt thereof.

19. A pharmaceutical composition comprising a compound of any one of claims 1-18, and a pharmaceutically acceptable carrier.

20. A method of treating a cannabinoid 1 receptor (CB1R)-mediated disease or condition in a subject in need of treatment thereof, the method comprising administering to said subject a therapeutically effective amount of a compound of any one of claims 1-18.

21. The method of claim 20, wherein the subject is a mammal, optionally a human.

22. The method of claim 20 or claim 21, wherein the disease or condition is selected from the group consisting of addiction, obesity, cancer, pain, female infertility, memory loss, cognitive dysfunction, Parkinson's disease, dyskinesia, tardive dyskinesia, Alzheimer's disease, amyotrophic lateral sclerosis (ALS), Tourette's Syndrome, stroke, atherosclerosis, hypotension, intestinal hypoactivity in paralytic ileus, inflammation, osteoporosis, hypercholesterolemia, dyslipidemia, diabetes, retinopathy, glaucoma, anxiety, depression and other mood disorders, gastrointestinal disorders, and metabolic disorders.

23. The method of claim 22, wherein the disease is obesity or addiction, optionally wherein the addiction is selected from cocaine addiction, opioid addiction, amphetamine addiction, cannabinoid addition, tobacco addiction, and alcohol addiction.

24. A method for inhibiting substance abuse, addiction, addictive behavior, or a symptom, behavior, or condition associated with addiction, the method comprising administering to a subject in need thereof a therapeutically effective amount of a compound of any one of claims 1-18.

25. The method of claim 24, wherein the addiction is selected from cocaine addiction, opioid addiction, amphetamine addiction, cannabinoid addition, tobacco addiction, and alcohol addiction.

26. The method of claim 24 or claim 25, wherein the administration prevents or inhibits relapse.

27. A method of modulating the activity of cannabinoid 1 receptor (CB1R), wherein the method comprises contacting a sample comprising CB1R with a compound of one of claims 1-18.

28. A compound of any one of claims 1-18, for use in medicine.

29. A compound of any one of claims 1-18, for the manufacture of a medicament for the treatment of one or more diseases or disorder of addiction, obesity, cancer, pain, female infertility, memory loss, cognitive dysfunction, Parkinson's disease, dyskinesia, tardive dyskinesia, Alzheimer's disease, amyotrophic lateral sclerosis (ALS), Tourette's Syndrome, stroke, atherosclerosis, hypotension, intestinal hypoactivity in paralytic ileus, inflammation, osteoporosis, hypercholesterolemia, dyslipidemia, diabetes, retinopathy, glaucoma, anxiety, depression and other mood disorders, gastrointestinal disorders, and metabolic disorders.

30. The compound according to claim 29, wherein the disease or disorder is obesity or addiction, optionally wherein the addiction is selected from cocaine addiction, opioid addiction, amphetamine addiction, cannabinoid addition, tobacco addiction, and alcohol addiction.

31. Use of a compound of any one of claims 1-18, for the treatment of one or more diseases or disorders of addiction, obesity, cancer, pain, female infertility, memory loss, cognitive dysfunction, Parkinson's disease, dyskinesia, tardive dyskinesia, Alzheimer's disease, amyotrophic lateral sclerosis (ALS), Tourette's Syndrome, stroke, atherosclerosis, hypotension, intestinal hypoactivity in paralytic ileus, inflammation, osteoporosis, hypercholesterolemia, dyslipidemia, diabetes, retinopathy, glaucoma, anxiety, depression and other mood disorders, gastrointestinal disorders, and metabolic disorders.

32. The use of claim 31, wherein the disease or disorder is obesity or addiction, optionally wherein the addiction is selected from cocaine addiction, opioid addiction, amphetamine addiction, cannabinoid addition, tobacco addiction, and alcohol addiction.
